(19) United States
(12) Patent Application Publication
(10) Pub. No.: US 2023/0140613 A1
(43) Pub. Date: May 4, 2023

(54) COLORIMETRIC SYSTEM FOR DETECTION OF COVID-19 USING EXHALED BREATH METABOLITES

(71) Applicants: Hasan Bagheri, Tehran (IR); Mohammad Mahdi Bordbar, Fasa (IR); Hosein Khoshsafar, Tehran (IR); Pegah Hashemi, Tehran (IR); Mostafa Ghanei, Tehran (IR)

(72) Inventors: Hasan Bagheri, Tehran (IR); Mohammad Mahdi Bordbar, Fasa (IR); Hosein Khoshsafar, Tehran (IR); Pegah Hashemi, Tehran (IR); Mostafa Ghanei, Tehran (IR)

(73) Assignees: Hasan Bagheri, Tehran (IR); Mohammad Mahdi Bordbar, Fasa (IR); Hosein Khoshsafar, Tehran (IR); Pegah Hashemi, Tehran (IR); Mostafa Ghanei, Tehran (IR); Farin Behbood Tashkhis Company, Tehran (IR); Bagiyatallah University of Medical Sciences - Chemical Injuries Research Center, Tehran (IR)

(21) Appl. No.: 18/090,964

(22) Filed: Dec. 29, 2022

Related U.S. Application Data
(60) Provisional application No. 63/325,162, filed on Mar. 30, 2022.

(51) Int. Cl. G01N 21/25; G01N 21/78; G01N 33/52

(52) U.S. Cl. CPC G01N 21/251; G01N 21/78; G01N 33/52

(57) ABSTRACT

A system for detecting COVID-19 infection, including a colorimetric sensor, an image-capturing device, and a processing unit. The colorimetric sensor includes an array of chemical receptors configured to be exposed to exhaled breath of a person suspected to be infected by COVID-19 virus. The array of chemical receptors includes an array of metalloporphyrazines, an array of organic dyes, an array of metalloporphyrins, an array of metal ion complexes, and an array of functionalized gold nanoparticles (AuNPs). The processing unit is configured to perform a method utilizing the image-capturing device. The method includes capturing a first image from the array of chemical receptors before exposure to exhaled breath, capturing a second image from the array of chemical receptors after exposure to exhaled breath, and detecting COVID-19 infection status of the person by analyzing color changes of the array of chemical receptors in the second image respective to the first image.

### CROSS-REFERENCE TO RELATED APPLICATION

This application claims the benefit of priority from pending U.S. Provisional Patent Application Ser. No. 63/325,162 filed on Mar. 30, 2022, and entitled “A COLORIMETRIC SNIFFER FOR QUALITATIVE AND QUANTITATIVE DETECTION OF COVID-19 DISEASE”, which is incorporated herein by reference in its entirety.

### TECHNICAL FIELD

The present disclosure generally relates to detection of a disease by analyzing metabolites in exhaled breath of a person using a colorimetric sensor, and particularly, to a method and device for detection of COVID-19 infection utilizing a colorimetric sensor with an array of chemical receptors interactive with chemical markers of COVID-19 infection in exhaled breath.

### BACKGROUND

Exhaled breath is composed of many volatile metabolites, which are classified into amines, ketones, acids, aldehydes, and carbohydrates. A variation in a metabolite concentration may be an indicator of a physiological disorder in human body, eventually leading to lung, kidney, and gastrointestinal diseases, airway inflammation, and metabolic disorders (e.g., diabetes, obesity, nonalcoholic fatty liver disease, and hyperlipidemia). So far, breath metabolites have been estimated using standard laboratory methods such as gas chromatography (GC), spectroscopic methods including mass spectrometry (MS), proton transfer reaction mass spectrometry (PTR-MS), laser spectroscopy, ion mobility spectrometry, and a combined approach of GC and MS. These methods provide sufficient information about type and amount of metabolites in infected, healthy, and even cured patients. Different metabolic profiles for each exhaled breath sample arise from appearance, removal, and a concentration change of a certain metabolite.

By the advent of SARS-CoV-2 in 2019, and the widespread epidemic COVID-19 as a contagious respiratory disease, several studies have been performed to evaluate and compare exhaled breath compositions of COVID-19 patients, healthy people, and individuals infected by other acute respiratory diseases. In this regard, values of some chemical compounds such as 2,4-octadiene 1-chloroheptane, nonanal, ethanal, octanal, acetone, butanone, and methanol in metabolic profiles of patients infected by COVID-19 have been reported to be different from those by other respiratory disorders. Discrimination of healthy samples from COVID-19 patients were obtained by evaluating changes in concentration of some volatile species, consisting of 1-propanol, 3,6-methylundecane, camphene, beta-cubebene, and iodobenzene. Determination of trace amounts of volatiles, and creation of a unique and reliable response for each exhaled breath sample are the most important features among conventional methods. Nevertheless, collecting virus-infected samples, and transferring them to an isolated clinical laboratory is a cumbersome process using conventional methods. Moreover, conventional methods suffer from the need to use of large, expensive, and complex instruments that require skilled personnel to set them up. Consumption of an excessive volume of materials or reagents, as well as lack of rapid responses is considered another limitation of conventional methods. As an alternative, electronic nose (e-nose) systems have been proposed to overcome limitations of conventional methods in analysis of breath metabolites for discrimination between COVID-19 infected patients and healthy individuals. An e-nose system consists of an array of sensing elements, such as gold nanoparticles or metal oxide semiconductors embedded in an electrical circuit for discriminating between breath volatile compounds of healthy and patient volunteers. Having a simple design, portability, and possibility of on-site sampling are advantages of e-nose-based systems. Also, sensor response, derived from an alteration in electrical resistance of sensing elements, can be shown digitally to users. However, e-nose systems have some disadvantages such as their complex, delicate, and costly electrical circuits, which is an essential part of an electrochemical transducer in an e-nose systems required for measuring changes in a sensing parameter, for example, potential, current, conductance, resistance, etc. Furthermore, weak van der Waals interactions occur between metabolites and an array of detecting components of e-nose sensors. Additionally, relative humidity of reaction medium may have a negative effect on e-nose sensor responses as well.

There is, therefore, a need for a rapid, simple, accurate, and cost-effective device, system, and method for detecting COVID-19 infection by analyzing exhaled breath metabolites of a person suspected to be infected by COVID-19 virus. Furthermore, there is a need for a device, system, and method for analyzing breath sample of a person suspected to be COVID-19-infected with an on-site and non-invasive sampling of exhaled breath.

### SUMMARY

This summary is intended to provide an overview of the subject matter of the present disclosure, and is not intended to identify essential elements or key elements of the subject matter, nor is it intended to be used to determine the scope of the claimed embodiments. The proper scope of the present disclosure may be ascertained from the claims set forth below in view of the detailed description below and the drawings.

In one general aspect, the present disclosure describes an exemplary system for detecting COVID-19 infection. The system may include a colorimetric sensor, an image-capturing device, and a processing unit. In an exemplary embodiment, the colorimetric sensor may include an array of chemical receptors deposited on a respective array of individual areas of a hydrophilic paper substrate. In an exemplary embodiment, the array of chemical receptors may be configured to be exposed to exhaled breath of a person suspected to be infected by COVID-19 virus. In an exemplary embodiment, the array of chemical receptors may include an array of metalloporphyrazines solutions, an array of organic dyes solutions, an array of metalloporphyrins solutions, an array of metal ion complexes solutions, and an array of functionalized gold nanoparticles (AuNPs) dispersions. In an exemplary embodiment, the image-capturing device may be configured to capture an image from the array of chemical receptors. In an exemplary embodiment, the processing unit may be electrically connected to the image-capturing device. In an exemplary embodiment, the processing unit may include a memory having processor-readable instructions stored therein and a processor. In an exemplary embodiment, the processor may be configured to access the memory and execute the processor-readable instructions. In an exemplary embodiment, the processor may be configured to perform a method by executing the processor-readable instructions. In an exemplary embodiment, the method may include capturing a first image from the array of chemical receptors before exposure to exhaled breath utilizing the image-capturing device, capturing a second image from the array of chemical receptors after at least 75 minutes of exposure to exhaled breath utilizing the image-capturing device, and detecting a COVID-19 infection status of the person by analyzing color changes of the array of chemical receptors in the second image respective to the first image.

In an exemplary embodiment, each metalloporphyrazine solution of the array of metalloporphyrazines solutions may include an aqueous solution of one of tetramethyl tetra-3,4-pyridinoporphyrazinato cobalt(II) ([Co(3,4-tmtppa)]⁴⁺), tetramethyl tetra-2,3-pyridinoporphyrazinato copper(II) ([Cu(2,3-tmtppa)]⁴⁺), tetramethyl tetra-3,4-pyridinoporphyrazinato zinc(II) ([Zn(3,4-tmtppa)]⁴⁺), and tetramethyl tetra-3,4-pyridinoporphyrazinato iron(II) (Fe(2,3-tmppa)) with a concentration of 1.5 mg mL⁻¹.

In an exemplary embodiment, each metalloporphyrin solution of the array of metalloporphyrins solutions may include a solution of one of [meso-tetraphenylporphyrin] iron(III) chloride (Fe(III)TPPCl), meso-tetrakis(4-chlorophenyl) porphyrin-manganese(III) acetate (Mn(III)T(4-Cl)PP(OAC)), eso-tetrakis(4-hydroxyphenyl) porphyrin-manganese(III) acetate (Mn(III)T(4-OH)PP(OAC)), [meso-tetraphenylporphyrin]-copper(II) (Cu(II)TPP), and [meso-tetraphenylporphyrin]-tin(II) (Sn(II)TPP) with a concentration of 6 mg mL⁻¹ in ethanol.

In an exemplary embodiment, each organic dye solution of the array of organic dyes solutions may include an organic dye, and additive, and a solvent. In an exemplary embodiment, the organic dye may include at least one of bromophenol red, bromocresol purple, acridine orange, indigo carmine, toluidine blue, malachite green, phenol red, pararosaniline, thymol blue, methyl red, bromophenol blue, bromopyrogallol red, methyl blue, and combinations thereof. In an exemplary embodiment, the additive may include at least one of DWES, phenylboronic acid (PBA), p-toluenesulfonic acid monohydrate (TsOH), tetrabutylammonium hydroxide (TBAOH), and combinations thereof. In an exemplary embodiment, DWES may be a mixture of 2,4-dinitrophenylhydrazine, deionized water, Ethanol (EtOH), and H₂SO₄. In an exemplary embodiment, the solvent may include at least one of water, ethanol, and combinations thereof. In an exemplary embodiment, a mixture of the organic dye and the additive with volume ratio of organic dye:additive equal to 4:1 may be dissolved in the solvent.

In an exemplary embodiment, each functionalized AuNPs dispersion of the array of functionalized AuNPs dispersions may include 3 mg mL⁻¹ of a plurality of AuNPs functionalized with at least one of bovine serum albumin (BSA), gallic acid (GA), polyvinyl pyrrolidone (PVP), and combinations thereof dispersed in deionized water.

In an exemplary embodiment, each metal ion complex solution of the array of metal ion complex solution may include a solution of at least one metal ion complex with a concentration of 0.01 mol L⁻¹ in a buffer solution with pH value of 9.0. In an exemplary embodiment, the at least one metal ion complex may include a complex of pyrocatechol violet (Py) with at least one of V (IV) ions, Fe (III) ions, Fe (II) ions, Cu (II) ions, Ni (II) ions, and combinations thereof.

In an exemplary embodiment, analyzing color changes of the array of chemical receptors in the second image respective to the first image may include generating a first color value vector associated with the first image, generating a second color value vector associated with the second image, generating a difference color value vector by subtracting each color value of the first color value vector from a respective color value of the second color value vector, and detecting the COVID-19 infection status of the person based on the difference color value vector. In an exemplary embodiment, the first color value vector may include a first array of a respective first set of three numerical color values of three respective color components of color of each respective chemical receptor of the array of chemical receptors in the first image. In an exemplary embodiment, the three-color components may include red, green, and blue. In an exemplary embodiment, the second color value vector may include a second array of a respective second set of three numerical color values respective to the three-color components of color of each respective chemical receptor of the array of chemical receptors in the second image.

In an exemplary embodiment, each of the first color value vector and the second color value vector is defined by a relation as follows:

Vⱼ=[R₁ⱼG₁ⱼBᵢⱼ . . . RᵢⱼGᵢⱼBᵢⱼ . . . RₙⱼGₙⱼBₙⱼ],

Where, j is equal to 1 for the first image and is equal to 2 for the second image, Rᵢⱼ is red component value of color of i^{th} chemical receptor of the array of chemical receptors in the j^{th} image, Gᵢⱼ is green component value of color of i^{th} chemical receptor of the array of chemical receptors in the j^{th} image, Bᵢⱼ is blue component value of color of i^{th} chemical receptor of the array of chemical receptors in the j^{th} image, and n comprises total number of chemical receptors of the array of chemical receptors.

In an exemplary embodiment, the difference color value vector may be defined by a relation as follows:

ΔV=[ΔR₁ΔG₁ΔB₁ . . . ΔRᵢΔGᵢΔBᵢ . . . ΔRₙΔGₙΔBₙ],

Where, ΔRᵢ is defined by a relation of ΔRᵢ=Rᵢ₂−Rᵢ₁, ΔGᵢ is defined by a relation of ΔGᵢ=Gᵢ₂−Gᵢ₁, and ΔBᵢ is defined by a relation of ΔBᵢ=Bᵢ₂−Bᵢ₁.

In an exemplary embodiment, detecting the COVID-19 infection status of the person based on the difference color value vector may include calculating a magnitude of the difference color value vector and detecting the COVID-19 infection status of the person. In an exemplary embodiment, calculating the magnitude of the difference color value vector may be done using a relation defined as follows:

|ΔV|=\sqrt{Σᵢ₌₁ⁿ(ΔRᵢ)²+(ΔGᵢ)²+(ΔBᵢ)²},

Where, |ΔV| is the magnitude of the difference color value vector. In an exemplary embodiment, detecting the COVID-19 infection status of the person may include detecting the person is healthy if the magnitude of the difference color value vector is less than a threshold value or detecting the person is COVID-19 infected if the magnitude of the difference color value vector is more than the threshold value. In an exemplary embodiment, the threshold value may include a value of 375.17.

In an exemplary embodiment, detecting the COVID-19 infection status of the person based on the difference color value vector may include comparing each element of the difference color value vector with a respective element of a set of reference difference color value vectors and detecting the person being one of a COVID-19 infected patient, a healthy individual, or a cured individual after an infection with COVID-19 virus. In an exemplary embodiment, an exemplary set of reference difference color value vectors may include a first reference difference color value vector including a mean difference color value vector of a first plurality of difference color value vectors generated by exposing the colorimetric sensor to exhaled breath of a respective plurality of COVID-19 patients, a second reference difference color value vector including a mean difference color value vector of a second plurality of difference color value vectors generated by exposing the colorimetric sensor to exhaled breath of a respective plurality of healthy individuals, and a third reference difference color value vector including a mean difference color value vector of a third plurality of difference color value vectors generated by exposing the colorimetric sensor to exhaled breath of a respective plurality of cured individuals after a COVID-19 infection. In an exemplary embodiment, detecting the person being one of a COVID-19 infected patient, a healthy individual, or a cured individual after an infection with COVID-19 virus may include one of detecting the person is a COVID-19 infected patient if a difference percentage between each element of the difference color value vector and the respective element of the first reference difference color value vector is less than 5%, detecting the person is a healthy individual if a difference percentage between each element of the difference color value vector and the respective element of the second reference difference color value vector is less than 5%, or detecting the person is a cured individual after a COVID-19 infection if a difference percentage between each element of the difference color value vector and the respective element of the third reference difference color value vector is less than 5%. In an exemplary embodiment, the method may further include generating the set of reference difference color value vectors. In an exemplary embodiment, generating the set of reference difference color value vectors may include generating a set of three pluralities of difference color value vectors by exposing the colorimetric sensor to exhaled breath of three respective groups and forming a respective reference difference color value vector for each plurality of difference color value vectors of three pluralities of difference color value vectors by calculating an average of respective elements of each plurality of difference color value vectors. In an exemplary embodiment, the three groups may include a plurality of COVID-19 patients, a plurality of healthy individuals, and a plurality of cured individuals after a COVID-19 infection.

In an exemplary embodiment, detecting the COVID-19 infection status of the person may include detecting the person is infected by COVID-19 virus if a change in color of at least one chemical receptor of a set of COVID-19 indicative chemical receptors is detected in the second image respective to the first image. In an exemplary embodiment, the set of COVID-19 indicative chemical receptors may include an aqueous solution of [Co(3,4-tmtppa)]⁴⁺ with a concentration of 1.5 mg·mL⁻¹, a solution of a mixture of indigo carmine and DWES in water with a volume ratio of indigo carmine:DWES equal to 4:1, a solution of a mixture of phenol red and DWES in ethanol with a volume ratio of phenol red:DWES equal to 4:1, a solution of a mixture of methyl red and TBAOH in ethanol with a volume ratio of methyl red:TBAOH equal to 4:1, a solution of Fe(III)TPPCl with a concentration of 6 mg mL⁻¹ in ethanol, and a solution of a mixture of bromocresol purple and PBA in ethanol with a volume ratio of bromocresol purple:PBA equal to 4:1.

In an exemplary embodiment, detecting the COVID-19 infection status of the person may include detecting the person is healthy if a change in color of at least one chemical receptor of a set of non-COVID-19 infection indicative chemical receptors is detected in the second image respective to the first image. In an exemplary embodiment, the set of non-COVID-19 infection indicative chemical receptors may include an aqueous solution of [Cu(2,3-tmtppa)]⁴⁺ with a concentration of 1.5 mg·mL⁻¹, a solution of a mixture of toluidine blue and DWES in water with a volume ratio of toluidine blue:DWES equal to 4:1, and a solution of a mixture of pararosaniline and TsOH in ethanol with a volume ratio of pararosaniline:TsOH equal to 4:1.

In an exemplary embodiment, detecting the COVID-19 infection status of the person may further include detecting a severity grade of COVID-19 infection of the person. In an exemplary embodiment, detecting the severity grade of COVID-19 infection of the person may be carried out by analyzing color changes of at least one severity indicator of a set of COVID-19 severity indicators of the array of chemical receptors in the second image respective to the first image. In an exemplary embodiment, the set of COVID-19 severity indicators may include the solution of the mixture of methyl red and TBAOH in ethanol with the volume ratio of methyl red:TBAOH equal to 4:1 and the solution of the mixture of bromocresol purple and PBA in ethanol with the volume ratio of bromocresol purple:PBA equal to 4:1.

In an exemplary embodiment, detecting the severity grade of COVID-19 infection of an exemplary person may include extracting two sets of three numerical color values of the at least one severity indicator in the first image and the second image, generating a difference color value vector associated with the at least one severity indicator by subtracting the two sets of three numerical color values from each other, calculating a magnitude of discoloration of the at least one severity indicator in the second image respective to the first image, and detecting severity grade of COVID-19 infection of the person based on the calculated magnitude of discoloration of the at least one severity indicator in the second image respective to the first image. In an exemplary embodiment, the three numerical color values may include respective values of three-color components of color of the at least one severity indicator. In an exemplary embodiment, the three-color components may include red, green, and blue.

In an exemplary embodiment, calculating the magnitude of discoloration of the at least one severity indicator in the second image respective to the first image may include calculating magnitude of the difference color value vector associated with the at least one severity indicator. In an exemplary embodiment, the magnitude of the difference color value vector associated with the at least one severity indicator may be calculated using a relation defined by:

|ΔV|=\sqrt{(ΔR)²+(ΔG)²+(ΔB)²},

Where, |ΔV| is the magnitude of discoloration of the at least one severity indicator, ΔR is a difference between respective red color values in the first image and the second image, ΔG is a difference between respective green color values in the first image and the second image, and ΔB is a difference between respective blue color values in the first image and the second image.

In an exemplary embodiment, detecting the severity grade of COVID-19 infection of an exemplary person based on the calculated magnitude of discoloration of the at least one severity indicator in the second image respective to the first image may include one of detecting an exemplary person is mildly infected by COVID-19 virus, detecting an exemplary person is moderately infected by COVID-19 virus, detecting an exemplary person is severely infected by COVID-19 virus, and detecting an exemplary person is highly-severe infected by COVID-19 virus. In an exemplary embodiment, detecting the severity grade of COVID-19 infection of an exemplary person may include detecting the person is mildly infected by COVID-19 virus if the magnitude of discoloration of the at least one severity indicator is in a range of 0 to 91. In an exemplary embodiment, a mildly infection by COVID-19 virus may include a cycle threshold (CT) number in a range of 27 to 30. In an exemplary embodiment, an exemplary CT number may include a CT number for N gene obtained in a polymerase-chain-reaction (PCR) test applied to an exemplary person. In an exemplary embodiment, detecting the severity grade of COVID-19 infection of an exemplary person may include detecting the person is moderately infected by COVID-19 virus if the magnitude of discoloration of the at least one severity indicator is in a range of 92 to 121. In an exemplary embodiment, a moderately infection by COVID-19 virus may include a CT number in a range of 24 to 26. In an exemplary embodiment, detecting the severity grade of COVID-19 infection of an exemplary person may include detecting the person is severely infected by COVID-19 virus if the magnitude of discoloration of the at least one severity indicator is in a range of 122 to 145. In an exemplary embodiment, a severely infection by COVID-19 virus may include a CT number in a range of 19 to 23. In an exemplary embodiment, detecting the severity grade of COVID-19 infection of an exemplary person may include detecting the person is highly-severe infected by COVID-19 virus if the magnitude of discoloration of the at least one severity indicator is in a range of 145 to 176. In an exemplary embodiment, a highly-severe infection by COVID-19 virus may include a CT number in a range of 15 to 18.

### BRIEF DESCRIPTION OF THE DRAWINGS

The drawing figures depict one or more embodiments in accord with the present teachings, by way of example only, not by way of limitation. In the figures, like reference numerals refer to the same or similar elements.

FIG. 1A illustrates a schematic view of an exemplary colorimetric sensor for detecting a disease using exhaled breath metabolites, consistent with one or more exemplary embodiments of the present disclosure.

FIG. 1B illustrates a schematic view of an exemplary attachable colorimetric sensing set, consistent with one or more exemplary embodiments of the present disclosure.

FIG. 1C illustrates a schematic view of an exemplary attachable colorimetric sensing set attached on an exemplary first face mask, consistent with one or more exemplary embodiments of the present disclosure.

FIG. 1D illustrates a schematic view of covering an exemplary attachable colorimetric sensing set attached on an exemplary first face mask by an exemplary second face mask, consistent with one or more exemplary embodiments of the present disclosure.

FIG. 1E illustrates another schematic view of covering an exemplary attachable colorimetric sensing set attached on an exemplary first face mask by an exemplary second face mask, consistent with one or more exemplary embodiments of the present disclosure.

FIG. 2 shows a schematic view of an exemplary system for detecting COVID-19 infection, consistent with one or more exemplary embodiments of the present disclosure.

FIG. 3 shows a flowchart of an exemplary method for detecting COVID-19 infection using exemplary exhaled breath of a person who is suspected to be infected by COVID-19 virus, consistent with one or more exemplary embodiments of the present disclosure.

FIG. 4 shows a flowchart of an exemplary method for detecting COVID-19 infection status of an exemplary person by analyzing color changes of an exemplary array of chemical receptors in an exemplary second image respective to an exemplary first image, consistent with one or more exemplary embodiments of the present disclosure.

FIG. 5 shows an exemplary computer system in which an embodiment of the present disclosure, or portions thereof, may be implemented as computer-readable code, consistent with one or more exemplary embodiments of the present disclosure.

FIG. 6 shows an exemplary pattern of an exemplary array of individual areas of an exemplary colorimetric sensor, consistent with one or more exemplary embodiments of the present disclosure.

FIG. 7 shows a chart of response of an exemplary colorimetric sensor using four models described in Table 1 for optimizing concentration of each exemplary chemical receptor, consistent with one or more exemplary embodiments of the present disclosure.

FIG. 8 shows a chart of response of an exemplary colorimetric sensor using five models described in Table 2 for optimizing ratio of organic dyes to additives, consistent with one or more exemplary embodiments of the present disclosure.

FIG. 9 shows a chart of response of an exemplary colorimetric sensor for different periods of time of incubation of exhaled breath and an exemplary array of chemical receptors, consistent with one or more exemplary embodiments of the present disclosure.

FIG. 10A shows a chart of score plots for classification of patients and healthy controls, obtained by PCA-LDA analysis using an exemplary fabricated colorimetric sensor with optimized array of chemical receptors compositions after 75 min exposure of an exemplary array of chemical receptors to exhaled breath of participants, consistent with one or more exemplary embodiments of the present disclosure.

FIG. 10B shows a chart of score plots for classification of patients and cured individuals, obtained by PCA-LDA analysis using an exemplary fabricated colorimetric sensor with optimized array of chemical receptors compositions after 75 min exposure of an exemplary array of chemical receptors to exhaled breath of participants, consistent with one or more exemplary embodiments of the present disclosure.

FIG. 10C shows a chart of score plots for classification of healthy controls and cured individuals, obtained by PCA-LDA analysis using an exemplary fabricated colorimetric sensor with optimized array of chemical receptors compositions after 75 min exposure of an exemplary array of chemical receptors to exhaled breath of participants, consistent with one or more exemplary embodiments of the present disclosure.

FIG. 11 shows a chart of magnitude of an exemplary difference color value vector for chemical receptor methyl red combined with TBAOH (methyl red+TBAOH) versus severity levels of COVID-19 infection, consistent with one or more exemplary embodiments of the present disclosure.

FIG. 12 shows a chart of magnitude of an exemplary difference color value vector for chemical receptor bromocresol purple combined with PBA (bromocresol purple+PBA) versus severity levels of COVID-19 infection, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary embodiments will be readily apparent to one skilled in the art, and the general principles defined herein may be applied to other embodiments and applications without departing from the scope of the present disclosure. The present disclosure is not intended to be limited to the embodiments shown, but is to be accorded the widest possible scope consistent with the principles and features disclosed herein.

The present disclosure is directed to exemplary systems and methods to detect a disease, or respiratory and body disorders of a person using exhaled breath metabolites of the person. In an exemplary embodiment, an infection with COVID-19 virus may be detected based on exemplary exhaled breath metabolites utilizing exemplary method and/or system disclosed herein. In an exemplary embodiment, smokers and people with kidney disease, lung disease, and diabetes may be identified based on exemplary exhaled breath metabolites utilizing exemplary method and/or system disclosed herein. In an exemplary embodiment, a severity of COVID-19 infection may be further detected utilizing exemplary method and/or system. In an exemplary embodiment, a COVID-19 status of a person may be detected as one of an infection with COVID-19 virus, a healthy status, or a cured status after a COVID-19 infection utilizing exemplary method and/or system disclosed herein. In an exemplary embodiment, exemplary system may include a colorimetric sensor, an image-capturing device, and a processing unit electrically connected to exemplary image-capturing device. In an exemplary embodiment, exemplary colorimetric sensor may include an array of chemical receptors deposited on a respective array of individual areas of a hydrophilic paper isolated from each other by a hydrophobic material. In an exemplary embodiment, an array of individual areas may include an array of bare parts of an exemplary hydrophilic paper and an exemplary hydrophobic material may be filled in texture of an exemplary hydrophilic paper in spaces among exemplary individual areas. In an exemplary embodiment, exemplary hydrophobic material may include a hydrophobic printing ink and may be configured to prevent spillover or penetration of a fluid (e.g., a chemical receptors) from one individual area of an exemplary array of individual areas to another. In an exemplary embodiment, an exemplary colorimetric sensor may be exposed to exhaled breath of a person suspected to be infected by COVID-19 virus and discoloration of one or more chemical receptors of an exemplary array of chemical receptor may be analyzed after a pre-determined period of time of interaction between exhaled breath metabolites and an exemplary array of chemical receptor. In an exemplary embodiment, an exemplary array of chemical receptors may discolor with a discriminative manner responsive to an interaction with specific metabolites of exemplary exhaled breath associated with a disease such as COVID-19 infection. In an exemplary embodiment, an exemplary array of chemical receptors may include a first plurality of chemical receptors being interactive with COVID-19 infection indicators of exhaled breath metabolites and a second plurality of chemical receptors being interactive with non-COVID-19-infection indicators of exhaled breath metabolites. In an exemplary embodiment, an exemplary array of chemical receptors may further include a third plurality of chemical receptors being interactive with cured status indicators of exhaled breath metabolites. In an exemplary embodiment, a first group of chemical receptors of an exemplary array of chemical receptors may discolor responsive to an interaction with a first group of metabolites, which are chemical markers of COVID-19 infection and are present in exemplary exhaled breath of a COVID-19 patient. In an exemplary embodiment, a second group of chemical receptors of an exemplary array of chemical receptors may discolor responsive to an interaction with a second group of metabolites, which are chemical markers of normal status and are present in an exemplary exhaled breath of a healthy individual. In an exemplary embodiment, a third group of chemical receptors of an exemplary array of chemical receptors may discolor responsive to an interaction with a third group of metabolites, which are chemical markers of cured COVID-19 infection and are present in an exemplary exhaled breath of a cured individual after a COVID-19 infection. As used herein, a “cured individual” may refer to a cured COVID-19-infected patient. In an exemplary embodiment, a “cured individual” may refer to a COVID-19-infected patient who had been treated and at least two months have passed from his/her recovery. In an exemplary embodiment, an image may be captured from an exemplary array of chemical receptors after a pre-determined period of time of interaction between exemplary exhaled breath and an exemplary array of chemical receptors. In an exemplary embodiment, an exemplary pre-determined period of time may include at least 75 minutes. In an exemplary embodiment, a COVID-19 infection status may be detected by comparing discoloration of an exemplary array of chemical receptors in an exemplary captured image after interaction with exhaled breath respective to an initial image captured from an exemplary array of chemical receptors before exposure to exhaled breath. In an exemplary embodiment, discoloration of an exemplary array of chemical receptors may be compared with discolorations recorded in a plurality of captured images from an exemplary array of chemical receptors after interaction with exhaled breath of a respective plurality of individuals with known status of COVID-19 infection. In an exemplary embodiment, a person may be detected to be infected with COVID-19 virus if discoloration of an exemplary array of chemical receptors after interaction with exemplary exhaled breath is in a range of discolorations of an exemplary array of chemical receptors after interaction with exhaled breath of a plurality of known COVID-19 patients. In an exemplary embodiment, a person may be detected to be healthy if discoloration of an exemplary array of chemical receptors after interaction with exemplary exhaled breath of the person is in a range of discolorations of an exemplary array of chemical receptors after interaction with exhaled breath of a plurality of known healthy people. In an exemplary embodiment, a person may be detected to be a COVID-19-cured individual if discoloration of an exemplary array of chemical receptors after interaction with exhaled breath of the person is in a range of discolorations of an exemplary array of chemical receptors after interaction with exhaled breath of a plurality of known cured individuals. In an exemplary embodiment, an array of chemical receptors may include five sets of chemical receptors including porphyrazines, organic dyes modified with additives, porphyrins, inorganic metal ion complexes, and functionalized gold nanoparticles.

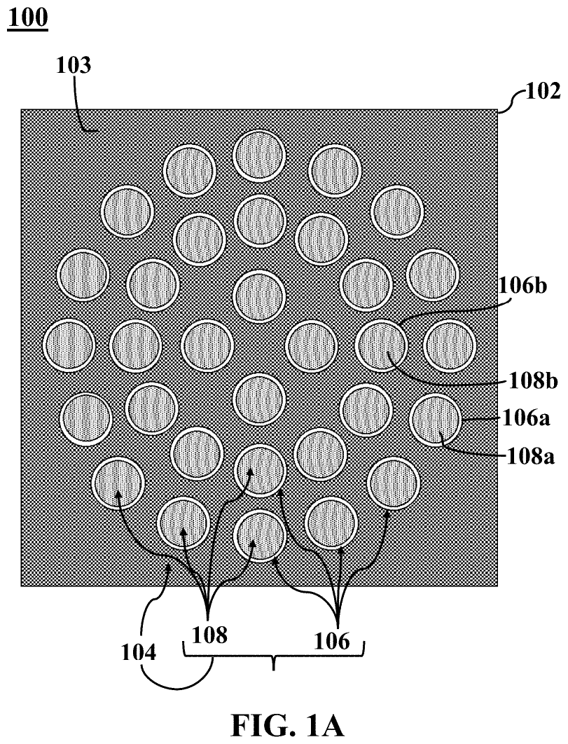

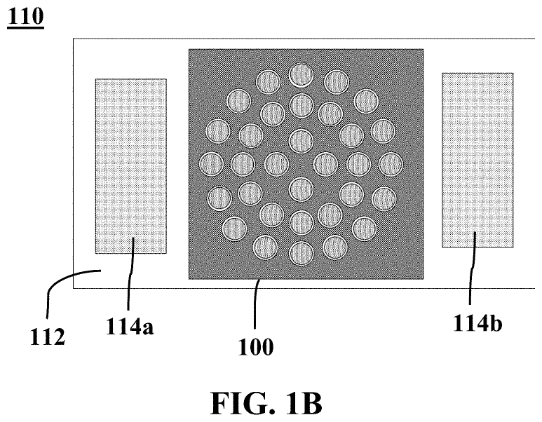

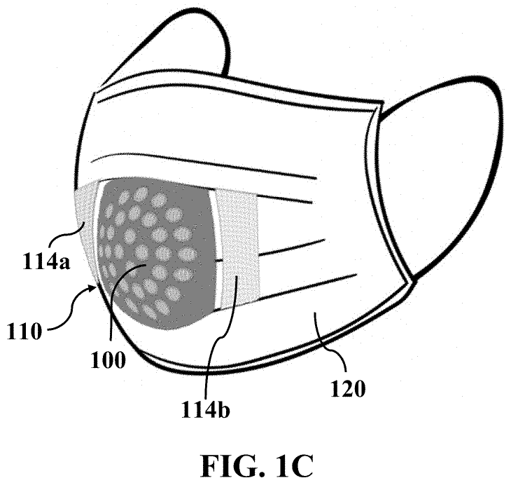

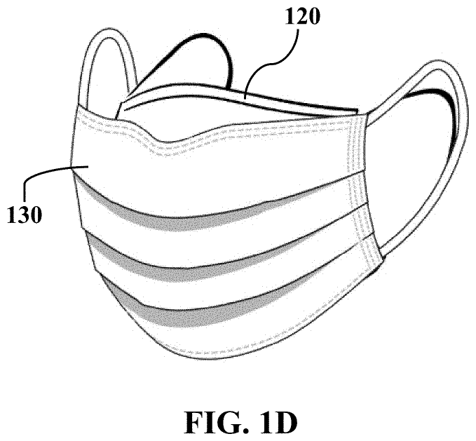

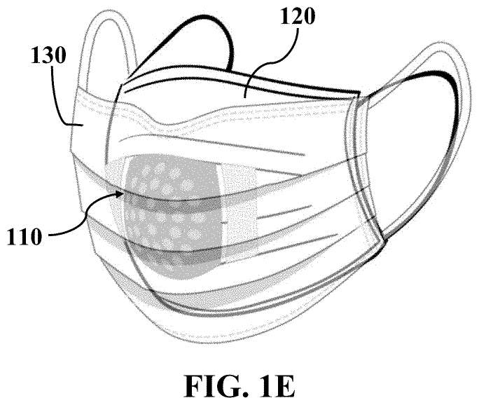

FIG. 1A illustrates a schematic view of a colorimetric sensor 100 for detecting a disease using exhaled breath metabolites, consistent with one or more exemplary embodiments of the present disclosure. In an exemplary embodiment, exemplary colorimetric sensor 100 may be utilized by system 200 and/or one or more steps of method 300 illustrated herein below. In an exemplary embodiment, exemplary colorimetric sensor 100 may be used to detect COVID-19 infection of a person using a COVID-19 biomarkers in exhaled breath of the person. In an exemplary embodiment, exemplary colorimetric sensor 100 may include an exemplary array of sensing zones 104 formed on an exemplary hydrophilic paper substrate 102. In an exemplary embodiment, hydrophilic paper substrate 102 may include a piece of a cellulosic paper. In an exemplary embodiment, hydrophilic paper substrate 102 may include a piece of a filter paper.

In an exemplary embodiment, exemplary colorimetric sensor 100 may further include a hydrophobic barrier 103 coated on parts of exemplary hydrophilic paper substrate 102 among array of sensing zones 104. In an exemplary embodiment, exemplary hydrophobic barrier 103 may filled within parts of texture of exemplary hydrophilic paper substrate 102 among array of sensing zones 104. In an exemplary embodiment, hydrophobic barrier 103 may be configured to isolate array of sensing zones 104 from each other by blocking pores of exemplary hydrophilic paper substrate 102 at spaces between each two sensing zone of array of sensing zones 104. In an exemplary embodiment, exemplary hydrophobic barrier 103 may include a hydrophobic material. In an exemplary embodiment, exemplary hydrophobic barrier 103 may include a hydrophobic printing ink penetrated into parts of texture of exemplary hydrophilic paper substrate 102 at spaces between each two sensing zone of array of sensing zones 104. In an exemplary embodiment, an exemplary hydrophobic printing ink may be printed on parts of an exemplary hydrophilic paper substrate 102 except sensing zones 104, and an exemplary printed hydrophobic printing ink may be penetrated into respective parts of texture of exemplary hydrophilic paper substrate 102 except sensing zones 104 by heating exemplary hydrophilic paper substrate 102.

In an exemplary embodiment, exemplary array of sensing zones 104 may include a respective array of chemical receptors 108 deposited (or coated) on a respective array of individual areas 106. For example, exemplary individual areas 106a and 106b of array of individual areas 106 may be coated with respective exemplary chemical receptors 108a and 108b of array of chemical receptors 108. In an exemplary embodiment, each respective individual area, for example, individual area 106a or 106b of exemplary array of individual areas 106 may have a circular shape. In an exemplary embodiment, exemplary hydrophilic paper substrate 102 may be divided into two parts consisting of exemplary array of individual areas 106 and an area among array of individual areas 106 coated with exemplary hydrophobic barrier 103. In an exemplary embodiment, array of individual areas 106 may include a plurality of circular bare parts of exemplary hydrophilic paper substrate 102. In an exemplary embodiment, exemplary array of individual areas 106 may include a plurality of individual bare parts of an exemplary hydrophilic paper substrate 102 separated (isolated) from each other by exemplary hydrophobic barrier 103. In an exemplary embodiment, exemplary hydrophobic barrier 103 may include a hydrophobic material coated on parts of exemplary hydrophilic paper substrate 102 except exemplary array of individual areas 106. In an exemplary embodiment, exemplary hydrophobic barrier 103 may include a hydrophobic material coated on parts of exemplary hydrophilic paper substrate 102 among exemplary array of individual areas 106. In an exemplary embodiment, exemplary hydrophobic barrier 103 may include a hydrophobic material filled within parts of texture of exemplary hydrophilic paper substrate 102 between each two individual areas (e.g., individual areas 106a and 106b) of exemplary array of individual areas 106. In an exemplary embodiment, exemplary hydrophobic barrier 103 may prevent penetration or spillover of a fluid from an exemplary individual area (e.g., as individual area 106a) to parts of hydrophilic paper substrate 102 among array of individual areas 106 or to another individual area (e.g., as individual area 106b) due to hydrophobicity of hydrophobic barrier 103. For example, chemical receptor 108a may not spillover or penetrate from individual area 106a to another individual area, for example, individual area 106b of array of individual areas 106 so that exemplary individual area 106a with exemplary chemical receptor 108a thereon may be isolated from a surrounding area on hydrophilic paper substrate 102. In an exemplary embodiment, exemplary hydrophobic barrier 103 may prevent any perturbation between responses of each two sensing zone of exemplary array of sensing zones 104 by isolating exemplary array of sensing zones 104 from each other.

In an exemplary embodiment, an exemplary chemical receptor 108a of array of chemical receptors 108 may include a chemical receptor of a respective biomarker of COVID-19 infection in exhaled breath metabolites. In an exemplary embodiment, an exemplary chemical receptor 108a of array of chemical receptors 108 may discolor due to an interaction with a metabolite of exhaled breath. In an exemplary embodiment, an exemplary chemical receptor 108a of array of chemical receptors 108 may include a chemical compound having a color reagent. In an exemplary embodiment, color of an exemplary chemical receptor 108a of exemplary array of chemical receptors 108 may change due to an interaction between an exemplary chemical receptor 108a and an analyte in exhaled breath after exposure of an exemplary chemical receptor 108a to exhaled breath. In an exemplary embodiment, an exemplary analyte may include a chemical marker of a disease in exhaled breath of a person suspected to have an exemplary disease. In an exemplary embodiment, an exemplary chemical receptor 108a of exemplary array of chemical receptors 108 may include a chemical compound (composition) capable of recoloring by interacting with a chemical marker of COVID-19 in exhaled breath. In an exemplary embodiment, an exemplary chemical marker of COVID-19 may include a chemical compound in exhaled breath having different amounts in exhaled breath of a COVID-19 infected patient and exhaled breath of a healthy individual. In an exemplary embodiment, color of an exemplary chemical compound (composition) of exemplary chemical receptor 108a may change due to chemically interacting with an exemplary chemical in exhaled breath with an amount of more than a threshold.

In an exemplary embodiment, changes of volatile chemical compounds in exhaled breath may be discriminative factor between patients infected by COVID-19 virus and healthy individuals, and other people with non-COVID lung diseases. In an exemplary embodiment, exemplary volatile chemical compounds may be classified to alkanes, alkenes, aldehydes, ketones, acids, alcohols, and arenes categories. In an exemplary embodiment, array of chemical receptors 108 may interact with exemplary volatile chemical compounds through at least one of Lewis acid-base, Bronsted acid-base, electrostatic, H-bonding, charge transfer, π-π, dipole-dipole, hydrophobic interactions, and combinations thereof; thereby, a change in color of one or more chemical receptor of array of chemical receptors 108 may be occurred. In an exemplary embodiment, color changes of array of chemical receptors 108 may be detected with different intensities depending on type and amount of volatile chemical compounds.

In an exemplary embodiment, exemplary array of chemical receptors 108 may include several arrays of chemical receptors; allowing for providing chemical receptors sensitive to all categories of volatile chemical compounds as indicative factors of COVID-19 infection. In an exemplary embodiment, utilizing a wide range of different groups of chemical receptors and additionally, using multiple chemical receptors of each group with different reactivities with chemical markers of COVID-19 infection may result in enhancing detection ability of colorimetric sensor 100 in the presence of ultra-low amounts of chemical markers associated with COVID-19 infection in exemplary exhaled breath and increasing an efficiency of colorimetric sensor 100 to differentiating between COVID-19 infected patients and healthy individuals. In an exemplary embodiment, an exemplary array of chemical receptors 108 may include at least one of an array of metalloporphyrazines solutions, an array of metalloporphyrins solutions, an array of organic dyes solutions, an array of metal ion complexes solutions, an array of nanoparticles (NPs) dispersions, and combinations thereof.

In an exemplary embodiment, an exemplary array of metalloporphyrazines solutions may include an array of solutions of at least one water soluble tetramethyl quaternized tetracationic porphyrazine. In an exemplary embodiment, an exemplary array of metalloporphyrazines solutions may include at least one of tetramethyl tetra-3,4-pyridinoporphyrazinato cobalt(II) ([Co(3,4-tmtppa)]⁴⁺), tetramethyl tetra-2,3-pyridinoporphyrazinato copper(II) ([Cu(2,3-tmtppa)]⁴⁺), tetramethyl tetra-3,4-pyridinoporphyrazinato zinc(II) ([Zn(3,4-tmtppa)]⁴⁺), and tetramethyl tetra-3,4-pyridinoporphyrazinato iron(II) (Fe(2,3-tmppa)) dissolved in water. In an exemplary embodiment, each metalloporphyrazine solution of an exemplary array of metalloporphyrazines solutions may include an aqueous solution of one of [Co(3,4-tmtppa)]⁴⁺, [Cu(2,3-tmtppa)]⁴+, [Zn(3,4-tmtppa)]⁴+, and Fe(2,3-tmppa) with a concentration in a range of about 0.5 mg mL⁻¹ to about 2 mg mL⁻¹. In an exemplary embodiment, each metalloporphyrazine solutionan of an exemplary array of metalloporphyrazines solutions may include an aqueous solution of one of [Co(3,4-tmtppa)]⁴⁺, [Cu(2,3-tmtppa)]⁴⁺, [Zn(3,4-tmtppa)]⁴⁺, and Fe(2,3-tmppa) with a concentration of about 1.5 mg mL⁻¹. In an exemplary embodiment, each metalloporphyrazine solution of an exemplary array of metalloporphyrazines solutions may be deposited or spotted on a respective individual area of exemplary array of individual areas 106. In an exemplary embodiment, an exemplary array of metalloporphyrazines may tend to interact with volatile metabolites in exhaled breath having at least one of carboxylic, amino, and ketone groups. In an exemplary embodiment, an exemplary array of metalloporphyrazines may tend to participating in Lewis acids-bases adduct formation and being attached to analytes in exhaled breath by transferring non-bonding electron pairs. In an exemplary embodiment, color changes of an exemplary array of metalloporphyrazines in response to interactions with volatile metabolites in exhaled breath may be influenced by chemical properties of central metal, strict hindrance of macrocyclic aromatic structure, polarity, chemical hardness, and affinity of analytes. It should be noted that metalloporphyrazines contain meso nitrogen atoms, and have a high potential for participation in nucleophilic and H-bonding reactions. In other word, a presence of four electron acceptor pyridine rings in structure of metalloporphyrazines may increase Lewis acidity of metal centers of metalloporphyrazines, and consequently, increase metalloporphyrazines tendency for adduct formation by an exemplary analyte.

In an exemplary embodiment, an exemplary array of metalloporphyrins solutions may include at least one of [meso-tetraphenylporphyrin] iron(III) chloride (Fe(III)TPPCl), meso-tetrakis(4-chlorophenyl) porphyrin-manganese(III) acetate (Mn(III)T(4-Cl)PP(OAC)), eso-tetrakis(4-hydroxyphenyl) porphyrin-manganese(III) acetate (Mn(III)T(4-OH)PP(OAC)), [meso-tetraphenylporphyrin]-copper(II) (Cu(II)TPP), and [meso-tetraphenylporphyrin]-tin(II) (Sn(II)TPP) in ethanol. In an exemplary embodiment, each metalloporphyrin solution of an exemplary array of metalloporphyrins solutions may include a solution of one of Fe(III)TPPCl, Mn(III)T(4-Cl)PP(OAC), Mn(III)T(4-OH)PP(OAC), Cu(II)TPP, and Sn(II)TPP with a concentration in a range of about 2 mg mL⁻¹ to about 8 mg mL⁻¹ in ethanol. In an exemplary embodiment, each metalloporphyrin solution of an exemplary array of metalloporphyrins solutions may include a solution of one of Fe(III)TPPCl, Mn(III)T(4-Cl)PP(OAC), Mn(III)T(4-OH)PP(OAC), Cu(II)TPP, and Sn(II)TPP with a concentration of about 6 mg mL⁻¹ in ethanol. In an exemplary embodiment, each metalloporphyrin solution of an exemplary array of metalloporphyrin solutions may be deposited or spotted on a respective individual area of exemplary array of individual areas 106. In an exemplary embodiment, an exemplary array of metalloporphyrins may tend to interact with volatile compounds of exhaled breath having at least one of aldehyde, ketone, and amine functional groups. In an exemplary embodiment, an exemplary array of metalloporphyrins may tend to participating in Lewis acids-bases adduct formation and being attached to analytes by transferring non-bonding electron pairs. In an exemplary embodiment, a color change of metalloporphyrins in response to interactions with volatile metabolites in exhaled breath may be influenced by chemical properties of central metal, strict hindrance of macrocyclic aromatic structure, polarity, chemical hardness, and affinity of analytes.

In an exemplary embodiment, each organic dye solution of an exemplary array of organic dyes solutions may include an organic dye, an additive, and a solvent. In an exemplary embodiment, each organic dye solution of an exemplary array of organic dyes solutions may include a mixture of an exemplary organic dye and an exemplary additive dissolved in an exemplary solvent with a volume ratio of organic dye:additive in a range of 1:1 to 5:1. In an exemplary embodiment, each organic dye solution of an exemplary array of organic dyes solutions may include a mixture of an exemplary organic dye and an exemplary additive dissolved in an exemplary solvent with a volume ratio of organic dye:additive of 4:1. In an exemplary embodiment, an exemplary organic dye may include at least one of bromophenol red, bromocresol purple, acridine orange, indigo carmine, toluidine blue, malachite green, phenol red, pararosaniline, thymol blue, methyl red, bromophenol blue, bromopyrogallol red, methyl blue, and combinations thereof. In an exemplary embodiment, an additive may include at least one of DWES, phenylboronic acid (PBA), p-toluenesulfonic acid monohydrate (TsOH), tetrabutylammonium hydroxide (TBAOH), and combinations thereof. As used herein, “DWES” is a mixture of 2,4-dinitrophenylhydrazine, deionized water, ethanol, and H₂SO₄. In an exemplary embodiment, DWES is a mixture of 0.4 gr of 2,4-dinitrophenylhydrazine, 3.0 mL of deionized water, 10.0 mL of ethanol, and 2.0 mL of H₂SO₄. In an exemplary embodiment, an exemplary solvent may include at least one of water, ethanol, and combinations thereof. In an exemplary embodiment, water may be used as an exemplary solvent for preparation of an exemplary organic dye solution of an exemplary water-soluble organic dye. Furthermore, ethanol may be used as an exemplary solvent for preparation of an exemplary organic dye solution of an exemplary ethanol-soluble organic dye. In an exemplary embodiment, each organic dye solution of an exemplary array of organic dyes solutions may be deposited or spotted on a respective individual area of exemplary array of individual areas 106.

It should be noted that organic dyes are proton donors or acceptors whose color highly depends on pH of media in interaction with organic dyes. Herein, exemplary organic dyes may be mixed with exemplary additives to increase reactivity of organic dyes with exhaled breath metabolites. For example, each of exemplary additives DWES, PBA, TsOH, TBAOH, and combinations thereof may be added to an exemplary dye; thereby, allowing for increasing reactivity of an exemplary dye with one or more species of exhaled breath metabolites. In an exemplary embodiment, DWES may tend to do a nucleophilic attack to carbonyl species of exhaled breath metabolites, whereas PBA may bind to diols; both reactions leading to a change in concentration of H₃O⁺. In an exemplary embodiment, TsOH may provide conditions for interaction between aniline-containing dyes and aldehydes of exhaled breath metabolites. Moreover, TBAOH may be added to one or more organic dyes to induce facile proton transfer reactions between organic dyes and exhaled breath metabolites.

In an exemplary embodiment, an exemplary organic dye solution containing a mixture of one of bromophenol red, bromocresol purple, acridine orange, indigo carmine, toluidine blue, malachite green, phenol red, and pararosaniline with DWES may react with materials functionalized by aldehyde and/or keton groups. In an exemplary embodiment, an exemplary organic dye solution containing a mixture of pararosaniline and TsOH may be used to detect aldehyde containing volatiles due to a discoloration of mixture of pararosaniline and TsOH in response to interaction with aldehydes. In an exemplary embodiment, an exemplary organic dye solution containing a mixture of TBAOH with one of thymol blue, methyl red, and bromophenol blue may discolor in the presence of released protons during acid-base interactions. In an exemplary embodiment, an exemplary organic dye solution containing a mixture of PBA with one of bromocresol purple, bromopyrogallol red, and methyl blue may be used to detect diol-based materials.

In an exemplary embodiment, an exemplary array of NPs dispersions may include an array of functionalized gold nanoparticles (AuNPs) dispersions. In an exemplary embodiment, an exemplary array of functionalized AuNPs may include an array of AuNPs functionalized with at least one of bovine serum albumin (BSA), gallic acid (GA), polyvinyl pyrrolidone (PVP), and combinations thereof. In an exemplary embodiment, each functionalized AuNPs dispersion of an exemplary array of functionalized AuNPs dispersions may include a plurality of AuNPs functionalized with at least one of BSA, GA, PVP, and combinations thereof dispersed in deionized water with a concentration in a range of about 1 mg mL⁻¹ to about 4 mg mL⁻¹. In an exemplary embodiment, each functionalized AuNPs dispersion of an exemplary array of functionalized AuNPs dispersions may include a plurality of AuNPs functionalized with one of BSA, GA, and PVP dispersed in deionized water with a concentration of about 3 mg mL⁻¹. In an exemplary embodiment, each functionalized AuNPs dispersion of an exemplary array of functionalized AuNPs dispersions may be deposited or spotted on a respective individual area of exemplary array of individual areas 106.

In an exemplary embodiment, AuNPs may be used here as detecting elements for marker ions and chemical compounds of a disease, for example, COVID-19 infection due to AuNPs' specific properties, such as surface plasmon resonance, high surface-to-volume ratio, and molecular recognition properties. However, in an exemplary embodiment, an exemplary an array of AuNPs may be functionalized by an exemplary functionalizing agent, including at least one of BSA, GA, PVP, and combinations thereof, so that color of an exemplary array of functionalized AuNPs dispersions may selectively change responsive to an interaction with exhaled breath of a COVID-19 infected patient. In an exemplary embodiment, functional groups of an exemplary functionalizing agent (e.g., BSA, GA, and/or PVP), size of functionalized AuNPs, and electrical charge on surface of functionalized AuNPs may increase selectivity of functionalized AuNPs for detecting COVID-19 markers in exemplary exhaled breath. In an exemplary embodiment, an exemplary array of functionalized AuNPs dispersions may discolor in response to interaction with volatile compounds in exemplary exhaled breath containing electronegative atoms and ionic compounds. In an exemplary embodiment, an exemplary array of functionalized AuNPs dispersions may discolor in response to interaction with volatile compounds with a concentration of at least 10 ppb in exemplary exhaled breath.

In an exemplary embodiment, an exemplary array of metal ion complexes solutions may include an array of solutions of a respective array of metal ion complexes. In an exemplary embodiment, each metal ion complex of an exemplary array of metal ion complexes may include a complex of at least one organic dye and at least one metal ion. In an exemplary embodiment, an exemplary metal ion complex may include a metal ion chelated with an organic dye. In an exemplary embodiment, an exemplary metal ion complex may include a complex of pyrocatechol violet (Py) with at least one of V (IV) ions, Fe (III) ions, Fe (II) ions, Cu (II) ions, Ni (II) ions, and combinations thereof. In an exemplary embodiment, each metal ion complex solution of an exemplary array of metal ion complexes solutions may include a solution of an exemplary metal ion complex with a concentration of metal ion complex in a range of 0.001 mol L⁻¹ to 0.05 mol L⁻¹ in a buffer solution with pH value in a range 3.0 to 11.0. In an exemplary embodiment, an exemplary metal ion complex solution may include a solution of an exemplary metal ion complex with a concentration of 0.01 mol L⁻¹ in a buffer solution with pH value of 9.0. In an exemplary embodiment, an exemplary metal ion complex solution may be prepared by forming a mixture containing an organic dye, a metal ion solution, and a buffer solution. In an exemplary embodiment, an exemplary buffer solution may include a borate buffer solution with a concentration of about 0.1 M and pH of about 9.0. In an exemplary embodiment, an exemplary metal ion solution may include an aqueous solution of at least one of V (IV) ions, Fe (III) ions, Fe (II) ions, Cu (II) ions, Ni (II) ions, and combinations thereof. In an exemplary embodiment, each metal ion complex solution of an exemplary array of metal ion complexes solutions may be deposited or spotted on a respective individual area of exemplary array of individual areas 106. In an exemplary embodiment, an exemplary metal ion complex solution may be capable of detecting compounds containing carboxylic and amino functional groups in exemplary exhaled breath due to a discoloration of an exemplary metal ion complex solution while interacting with carboxylic and amino functional groups.

In an exemplary embodiment, an exemplary array of chemical receptors 108 may include 32 different chemical receptors coated on respective array of 32 individual areas 106. In an exemplary embodiment, an exemplary array of chemical receptors 108 may include 32 chemical receptors of [Co(3,4-tmtppa)]⁴⁺, [Zn(3,4-tmtppa)]⁴⁺, [Cu(2,3-tmtppa)]⁴⁺, Fe(2,3-tmppa), bromocresol red+DWES, bromocresol purple+DWES, acridine orange+DWES, indigo carmine+DWES, toluidine blue+DWES, malachite green+DWES, phenol red+DWES, pararosaniline+DWES, pararosaniline+TsOH, thymol blue+TBAOH, methyl red+TBAOH, bromophenol blue+TBAOH, Mn(III)T(4-Cl)PP(OAc), Fe(III)TPPCl, Mn(III)T(4-OH)PP(OAc), Cu(II)TPP, Sn(II)TPP, V(IV)-Py, and Fe(III)-Py, Fe(II)-Py, Cu(II)-Py, Ni(II)-Py, bromocresol purple+PBA, bromopyrogallol red+PBA, methyl blue+PBA, GA-AuNPs, BSA-AuNPs, and PVP-AuNPs.

In an exemplary embodiment of the present disclosure, a method for fabrication of exemplary colorimetric sensor 100 may be described. In an exemplary embodiment, an exemplary fabrication method of exemplary colorimetric sensor 100 may include drawing a pre-determined pattern of colorimetric sensor 100 in an image-drawing software, forming an exemplary array of individual areas 106 on an exemplary hydrophilic paper substrate 102 by printing the drawn pattern on exemplary hydrophilic paper substrate 102, forming an exemplary hydrophobic barrier 103 by heating an exemplary hydrophilic paper substrate 102 with the pattern printed thereon in an oven, and coating an exemplary array of chemical receptors 108 on respective array of individual areas 106 on an exemplary hydrophilic paper substrate 102. In an exemplary embodiment, an exemplary pre-determined pattern may include an array of individual circular zones arranged around a center of a rectangle. In an exemplary embodiment, the drawn pattern may be printed on an exemplary hydrophilic paper substrate 102 using a printer device. In an exemplary embodiment, an exemplary hydrophobic barrier 103 may be formed by heating an exemplary hydrophilic paper substrate 102 with the pattern printed thereon at a temperature of about 200° C. in an oven for a time period of about 45 minutes. In an exemplary embodiment, heating an exemplary hydrophilic paper substrate 102 with the pattern printed thereon may lead to melting printing ink, penetrating printing ink into texture of an exemplary hydrophilic paper substrate 102, and filling/blocking holes of an exemplary hydrophilic paper substrate 102; thereby, resulting in forming hydrophobic sites in printed sites of an exemplary printed pattern on an exemplary hydrophilic paper substrate 102. In an exemplary embodiment, coating an exemplary array of chemical receptors 108 on respective array of individual areas 106 on an exemplary hydrophilic paper substrate 102 may include filling each respective individual area of array of individual areas 106 with a respective chemical receptor of array of chemical receptors 108. In an exemplary embodiment, coating an exemplary chemical receptor (similar to exemplary chemical receptor 108a) on a respective individual area (similar to individual area 106a) may include filling a liquid-injecting instrument with an exemplary chemical receptor and injecting an exemplary chemical receptor onto a respective individual area of an exemplary array of individual areas 106. In an exemplary embodiment, an exemplary liquid-injecting instrument may include a syringe, a micropipette, and combinations thereof.

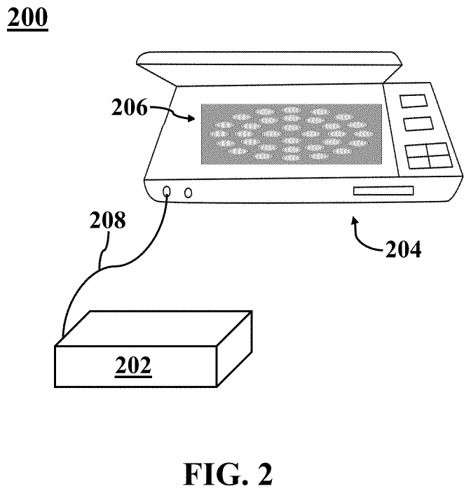

In an exemplary embodiment of the present disclosure, an exemplary colorimetric sensor 100 may be utilized in a system for detecting COVID-19 infection. FIG. 2 shows a schematic view of an exemplary system 200 for detecting COVID-19 infection, consistent with one or more exemplary embodiments of the present disclosure. Exemplary system 200 may include a processing unit 202, an image-capturing device 204, and a colorimetric sensor 206 similar to exemplary colorimetric sensor 100 illustrated in FIG. 1. In an exemplary embodiment, an exemplary colorimetric sensor 206 may be configured to be exposed to exhaled breath of a person suspected to be infected by COVID-19 virus and interact with exhaled breath metabolites in accordance with FIG. 1C described in details herein below. In an exemplary embodiment, exemplary image-capturing device 204 may be configured to capture images from an exemplary array of chemical receptors (similar to exemplary array of chemical receptors 108) of exemplary colorimetric sensor 206 before and after interaction with exhaled breath metabolites of an exemplary person. In an exemplary embodiment, image-capturing device 204 may be electrically connected to processing unit 202 via an electrically connection instrument 208. In an exemplary embodiment, electrically connection instrument 208 may include a wireless electrical connector or an electrically conductive wire. In an exemplary embodiment, image-capturing device 204 may be electrically connected to processing unit 202 using a wireless electrical connection utilizing Bluetooth modules embedded in processing unit 202 and image-capturing device 204. In an exemplary embodiment, image-capturing device 204 may be electrically connected to processing unit 202 using an electrically conductive wire. In an exemplary embodiment, image-capturing device 204 may include at least one of a camera, a digital camera, a camera of a cellphone, a scanner, and a paper-based document scanner. In an exemplary embodiment, processing unit 202 may be configured to receive a captured image of an exemplary array of chemical receptors of exemplary colorimetric sensor 206 form image-capturing device 204 and detecting a COVID-19 infection by analyzing an exemplary captured image. In an exemplary embodiment, processing unit 202 may include a memory having processor-readable instructions stored therein and a processor. An exemplary processor may be configured to access the memory and execute the processor-readable instructions. In an exemplary embodiment, the processor may be configured to perform a method by executing the processor-readable instructions. In an exemplary embodiment, an exemplary method may include one or more steps of an exemplary method 300 described herein below for detecting COVID-19 infection of a person using exemplary exhaled breath of an exemplary person.

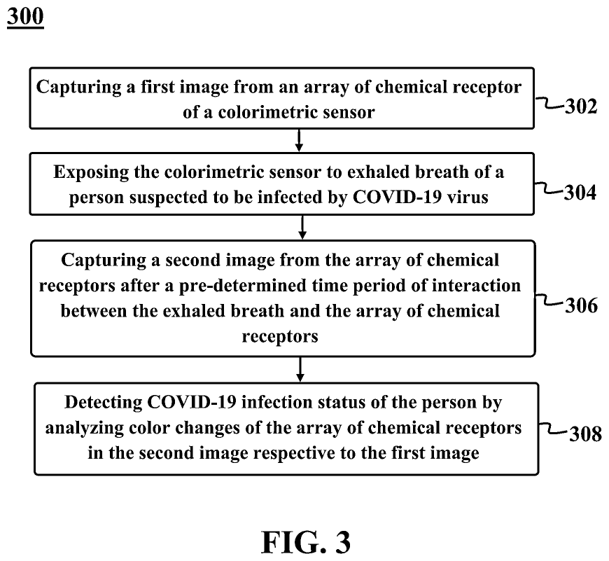

In an exemplary embodiment, the present disclosure describes an exemplary method for detecting COVID-19 infection. In an exemplary embodiment, an exemplary method may include detecting COVID-19 infection status of a person by analyzing exhaled breath of the person using exemplary system 200 utilizing an exemplary colorimetric sensor similar to colorimetric sensors 100 and 206. FIG. 3 shows a flowchart of an exemplary method 300 for detecting COVID-19 infection using exemplary exhaled breath of a person who is suspected to be infected by COVID-19 virus, consistent with one or more exemplary embodiments of the present disclosure. Exemplary method 300 may include capturing a first image from an exemplary array of chemical receptors 108 of an exemplary colorimetric sensor 100 (or 206) (step 302), exposing colorimetric sensor 100 (or 206) to exhaled breath of a person suspected to be infected by COVID-19 virus (step 304), capturing a second image from an exemplary array of chemical receptors 108 after a pre-determined time period of interaction between the exhaled breath and an exemplary array of chemical receptors 108 (step 306), and detecting a COVID-19 infection status of an exemplary person by analyzing color changes of an exemplary array of chemical receptors 108 in the second image respective to the first image (step 308). In an exemplary embodiment, one or more steps of exemplary method 300 may be carried out utilizing an exemplary system 200 described herein above. In an exemplary embodiment, exemplary steps of method 300 are described in further detail below in context of exemplary system 200 and colorimetric sensor 100 (or 206).

In further detail with respect to step 302, step 302 may include capturing a first image from an exemplary array of chemical receptors 108 of an exemplary colorimetric sensor 100 (or 206). In an exemplary embodiment, an exemplary first image may be captured from an exemplary array of chemical receptors 108 using image-capturing device 204 before exposure of an exemplary colorimetric sensor 100 (or 206) to exhaled breath.

In further detail with respect to step 304, step 304 may include exposing colorimetric sensor 100 (or 206) to exhaled breath of a person suspected to be infected by COVID-19 virus. In an exemplary embodiment, exposing colorimetric sensor 100 (or 206) to exhaled breath of an exemplary person may include directly exposure of colorimetric sensor 100 (or 206) to exhaled breath metabolites without need of an external force for collecting and transferring an exhaled breath sample to surface of colorimetric sensor 100 (or 206). In an exemplary embodiment, exposing colorimetric sensor 100 (or 206) to exhaled breath of an exemplary person may include placing an exemplary colorimetric sensor 100 (or 206) in front of mouth of an exemplary person. In an exemplary embodiment, exposing colorimetric sensor 100 (or 206) to exhaled breath of an exemplary person may include covering mouth of an exemplary person by a first face mask, attaching an exemplary colorimetric sensor 100 (or 206) on the first face mask in front of mouth of an exemplary person, and covering an exemplary colorimetric sensor 100 (or 206) by a second face mask. FIG. 1B illustrates a schematic view of an attachable colorimetric sensing set 110, consistent with one or more exemplary embodiments of the present disclosure. In an exemplary embodiment, an exemplary attachable colorimetric sensing set 110 may be used to expose an exemplary colorimetric sensor 100 (or 206) to exhaled breath of an exemplary person. In an exemplary embodiment, an exemplary attachable colorimetric sensing set 110 may include a strip 112, an exemplary colorimetric sensor 100 (or 206) pasted on exemplary strip 112, and two double-sided tapes 114a and 114b pasted on exemplary strip 112 on either side of exemplary colorimetric sensor 100 (or 206). In an exemplary embodiment, exemplary strip 112 may include a plastic strip made of a polymeric material, for example, poly(methyl methacrylate) (PMMA). FIG. 1C illustrates a schematic view of an exemplary attachable colorimetric sensing set 110 attached on a first face mask 120, consistent with one or more exemplary embodiments of the present disclosure. In an exemplary embodiment, an exemplary first face mask 120 may cover mouth of an exemplary person and an exemplary attachable colorimetric sensing set 110 may be attached on exemplary first face mask 120 using two double-sided tapes 114a and 114b so that an exemplary colorimetric sensor 100 (or 206) may be placed in front of mouth of an exemplary person. In an exemplary embodiment, an exemplary first face mask 120 may include a cloth mask having a plurality of pores with a size of less than about 5 μm for each respective pore. In an exemplary embodiment, an exemplary first face mask 120 may allow exhaled breath metabolites to pass there through, leading to an interaction between exhaled breath metabolites and an exemplary array of chemical receptors 108 of an exemplary colorimetric sensor 100 (or 206). In an exemplary embodiment, an exemplary first face mask 120 may further form a barrier in front of mouth of an exemplary person; allowing for preventing saliva droplets and metabolites being in contact with an exemplary colorimetric sensor 100 (or 206) so that only exhaled breath metabolites may be put in interaction with an exemplary array of chemical receptors 108 of an exemplary colorimetric sensor 100. FIG. 1D illustrates a schematic view of covering an exemplary attachable colorimetric sensing set 110 attached on a first face mask 120 by a second face mask 130, consistent with one or more exemplary embodiments of the present disclosure. Additionally, FIG. 1E illustrates another schematic view of covering an exemplary attachable colorimetric sensing set 110 attached on a first face mask 120 by a second face mask 130, consistent with one or more exemplary embodiments of the present disclosure. In an exemplary embodiment, an exemplary second face mask 130 may be used to cover an exemplary attachable colorimetric sensing set 110. In an exemplary embodiment, an exemplary second face mask 130 may form a protective layer on exemplary attachable colorimetric sensing set 110 against physical interference and environmental pollutions that may be caused due to a contact of exemplary attachable colorimetric sensing set 110 with a surrounding area, such as surrounding air. In an exemplary embodiment, an exemplary second face mask 130 may include a three-layer medical mask. In an exemplary embodiment, an exemplary colorimetric sensor 100 (or 206) may be exposed to exhaled breath of an exemplary person for a pre-determined period of time of at least 75 minutes to achieve a complete interaction of an exemplary array of chemical receptors 108 with exhaled breath metabolites. In an exemplary embodiment, color of one or more chemical receptors of an exemplary array of chemical receptors 108 may change due to an interaction between exhaled breath metabolites and an exemplary array of chemical receptors 108.

In further detail with respect to step 306, step 306 may include capturing a second image from an exemplary array of chemical receptors 108 after a pre-determined time period of interaction between the exhaled breath and an exemplary array of chemical receptors 108. In an exemplary embodiment, an exemplary second image may be captured from an exemplary array of chemical receptors 108 using image-capturing device 204 after interaction of exemplary exhaled breath metabolites with an exemplary array of chemical receptors 108 of an exemplary colorimetric sensor 100 (or 206).

In further detail with respect to step 308, step 308 may include detecting a COVID-19 infection status of an exemplary person by analyzing color changes of an exemplary array of chemical receptors 108 in an exemplary second image respective to an exemplary first image. In an exemplary embodiment, detecting COVID-19 infection status of an exemplary person by analyzing color changes of an exemplary array of chemical receptors 108 in an exemplary second image respective to an exemplary first image (step 308) may include comparing color changes of an exemplary array of chemical receptors 108 in an exemplary second image respective to an exemplary first image with color changes of an exemplary array of chemical receptors 108 in a plurality of images with known COVID-19 infection status respective to an exemplary first image. In an exemplary embodiment, the plurality of images with known COVID-19 infection status may include three sets of images associated with three groups of people with known COVID-19 infection status. In an exemplary embodiment, the three sets of images may include a first set of images associated with a respective plurality of COVID-19 infected patients, a second set of images associated with a respective plurality of healthy individuals, and a third set of images associated with a respective plurality of cured individuals after a COVID-19 infection. In an exemplary embodiment, each image of the plurality of images with known COVID-19 infection status may be captured and recorded after an interaction between an exemplary array of chemical receptors 108 and exhaled breath metabolites of each respective individual of people with known COVID-19 infection status.

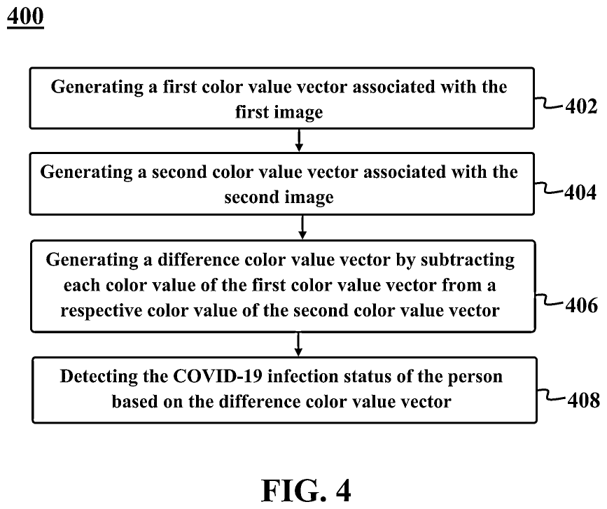

In more details with respect to step 308, detecting COVID-19 infection status of an exemplary person (step 308) may further include one of detecting an exemplary person being a COVID-19 infected patient, detecting an exemplary person being a healthy individual, and detecting an exemplary person being a cured individual after a COVID-19 infection. As used herein, a “cured individual” may refer to a cured COVID-19-infected patient. In an exemplary embodiment, a “cured individual” may refer to a COVID-19-infected patient who had been treated and at least two months passed from his/her recovery. In an exemplary embodiment, detecting COVID-19 infection status of an exemplary person (step 308) may include detecting that an exemplary person is a COVID-19 infected patient if color changes of an exemplary array of chemical receptors 108 in an exemplary second image respective to an exemplary first image is similar to color changes of an exemplary array of chemical receptors 108 in exemplary first set of images associated with the respective plurality of COVID-19 infected patients respective to an exemplary first image. In an exemplary embodiment, a similarity between color changes of an exemplary array of chemical receptors 108 in an exemplary second image respective to an exemplary first image and color changes of an exemplary array of chemical receptors 108 in exemplary first set of images associated with the respective plurality of COVID-19 infected patients respective to an exemplary first image may be analyzed by conducting an exemplary method 400 described below in further detail in context of FIG. 4. A “similarity” between color changes of an exemplary array of chemical receptors 108 in an exemplary second image respective to an exemplary first image and color changes of an exemplary array of chemical receptors 108 in an exemplary first set of images may refer to detecting discoloration of identical chemical receptors of an exemplary array of chemical receptors 108 in an exemplary second image and an exemplary first set of images. In an exemplary embodiment, detecting COVID-19 infection status of an exemplary person may include detecting an exemplary person is a COVID-19 infected patient if color of a first plurality of chemical receptors of exemplary array of chemical receptors 108 is changed in an exemplary second image respective to an exemplary first image. In an exemplary embodiment, an exemplary first plurality of chemical receptors of exemplary array of chemical receptors 108 may include [Co(3,4-tmtppa)]⁴⁺, indigo carmine+DWES, phenol red+DWES, methyl red+TBAOH, Fe(III)TPPCl, and bromocresol purple+PBA. In an exemplary embodiment, an exemplary first plurality of chemical receptors of exemplary array of chemical receptors 108 may further include bromocresol purple+DWES, malachite green+DWES, and thymol blue+TBAOH. In an exemplary embodiment, detecting COVID-19 infection status of an exemplary person (step 308) may include detecting that an exemplary person is a healthy individual if color changes of an exemplary array of chemical receptors 108 in an exemplary second image respective to an exemplary first image is similar to color changes of an exemplary array of chemical receptors 108 in exemplary second set of images associated with the respective plurality of healthy individuals respective to an exemplary first image. A “similarity” between color changes of an exemplary array of chemical receptors 108 in an exemplary second image respective to an exemplary first image and color changes of an exemplary array of chemical receptors 108 in an exemplary second set of images may refer to detecting discoloration of identical chemical receptors of an exemplary array of chemical receptors 108 in an exemplary second image and an exemplary second set of images. In an exemplary embodiment, a similarity between color changes of an exemplary array of chemical receptors 108 in an exemplary second image respective to an exemplary first image and color changes of an exemplary array of chemical receptors 108 in exemplary second set of images associated with the respective plurality of healthy individuals respective to an exemplary first image may be analyzed by conducting an exemplary method 400 described below in further detail in context of FIG. 4. In an exemplary embodiment, detecting COVID-19 infection status of an exemplary person may include detecting an exemplary person is a healthy individual if color of a second plurality of chemical receptors of exemplary array of chemical receptors 108 is changed in an exemplary second image respective to an exemplary first image. In an exemplary embodiment, an exemplary second plurality of chemical receptors of exemplary array of chemical receptors 108 may include [Cu(2,3-tmtppa)]⁴⁺, toluidine blue+DWES, and pararosaniline+TsOH. In an exemplary embodiment, an exemplary second plurality of chemical receptors of exemplary array of chemical receptors 108 may further include pararosaniline+DWES, Mn(III)T(4-Cl)PP(OAc), Mn(III)T(4-OH)PP(OAc), V(IV)-Py, and Fe(III)-Py. In an exemplary embodiment, detecting COVID-19 infection status of an exemplary person (step 308) may include detecting that an exemplary person is a cured individual after a COVID-19 infection if color changes of an exemplary array of chemical receptors 108 in an exemplary second image respective to an exemplary first image is similar to color changes of an exemplary array of chemical receptors 108 in an exemplary third set of images associated with the respective plurality of cured individuals respective to an exemplary first image. A “similarity” between color changes of an exemplary array of chemical receptors 108 in an exemplary second image respective to an exemplary first image and color changes of an exemplary array of chemical receptors 108 in an exemplary third set of images may refer to detecting discoloration of identical chemical receptors of an exemplary array of chemical receptors 108 in an exemplary second image and an exemplary third set of images. In an exemplary embodiment, a similarity between color changes of an exemplary array of chemical receptors 108 in an exemplary second image respective to an exemplary first image and color changes of an exemplary array of chemical receptors 108 in exemplary third set of images associated with the respective plurality of cured individuals respective to an exemplary first image may be analyzed by conducting an exemplary method 400 described below in further detail in context of FIG. 4. In an exemplary embodiment, detecting COVID-19 infection status of an person may include detecting an exemplary person is a cured individual if color of a third plurality of chemical receptors of exemplary array of chemical receptors 108 is changed in an exemplary second image respective to an exemplary first image. In an exemplary embodiment, an exemplary third plurality of chemical receptors of exemplary array of chemical receptors 108 may include bromocresol purple+DWES, malachite green+DWES, pararosaniline+DWES, thymol blue+TBAOH, Mn(III)T(4-Cl)PP(OAc), Mn(III)T(4-OH)PP(OAc), V(IV)-Py, and Fe(III)-Py.

In an exemplary embodiment, detecting COVID-19 infection status of an exemplary person by analyzing color changes of an exemplary array of chemical receptors 108 in an exemplary second image respective to an exemplary first image (step 308) may include extracting a respective set of color values associated with each chemical receptor in exemplary first and second images, generating color value vectors respective to exemplary first and second images using exemplary respective extracted sets of color values, generating a difference color value vector using color value vectors of exemplary first and second images, analyzing exemplary difference color value vector in comparison with a plurality of difference color value vectors formed for an exemplary respective plurality of images with known COVID-19 infection status, and detecting COVID-19 infection status of an exemplary person based on a similarity of an exemplary difference color value vector associated with an exemplary person with exemplary difference color value vectors associated with one group of three groups of people with known COVID-19 infection status. In an exemplary embodiment, analyzing exemplary difference color value vector in comparison with a plurality of difference color value vectors may be carried out using a principle component analysis-linear discriminate analysis (PCA-LDA) technique.

In an exemplary embodiment of the present disclosure, an exemplary method is described for detecting COVID-19 infection status of an exemplary person by analyzing color changes of an exemplary array of chemical receptors 108 in an exemplary second image respective to an exemplary first image (step 308) based on generating and utilizing an exemplary difference color value vector using an exemplary first image and an exemplary second image. FIG. 4 shows a flowchart of an exemplary method 400 for detecting COVID-19 infection status of an exemplary person by analyzing color changes of an exemplary array of chemical receptors 108 in an exemplary second image respective to an exemplary first image (step 308), consistent with one or more exemplary embodiments of the present disclosure. In an exemplary embodiment, exemplary method 400 may include generating a first color value vector associated with an exemplary first image (step 402), generating a second color value vector associated with an exemplary second image (step 404), generating a difference color value vector by subtracting each color value of an exemplary first color value vector from a respective color value of an exemplary second color value vector (step 406), and detecting a COVID-19 infection status of an exemplary person based on an exemplary difference color value vector (step 408).

In further detail with respect to step 402, step 402 may include generating a first color value vector associated with an exemplary first image. In an exemplary embodiment, an exemplary first color value vector may include a first array of a respective first set of three numerical color values of three respective color components of color of each respective chemical receptor of an exemplary array of chemical receptors 108 in an exemplary first image. In an exemplary embodiment, exemplary three-color components may include red, green, and blue. In an exemplary embodiment, generating an exemplary first color value vector may include extracting color values of blue, green, and red associated with each chemical receptor in an exemplary first image and forming an exemplary first array of exemplary respective first sets of extracted three color values respective to an exemplary array of chemical receptors 108. In an exemplary embodiment, extracting color values of blue, green, and red may be done using an image processing technique utilizing one or more processors. In an exemplary embodiment, extracting color values of blue, green, and red may be done using an image processing software, such as Image J or Adobe Photoshop utilizing one or more processors. In an exemplary embodiment, an exemplary first color value vector may be formed utilizing one or more processors. In an exemplary embodiment, an exemplary first color may be formed using a relation defined by Equation 1 as follows:

V₁=[R₁₁G₁₁B₁₁ . . . Rᵢ₁Gᵢ₁Bᵢ₁ . . . Rₙ₁Gₙ₁Bₙ₁], Equation 1

Where, Rᵢ₁ is red component value of color of i^{th} chemical receptor of an exemplary array of chemical receptors 108 in an exemplary first image, Gᵢ₁ is green component value of color of i^{th} chemical receptor of an exemplary array of chemical receptors 108 in an exemplary first image, Bᵢ₁ is blue component value of color of i^{th} chemical receptor of an exemplary array of chemical receptors 108 in an exemplary first image, and n is total number of chemical receptors of an exemplary array of chemical receptors 108.

In further detail with respect to step 404, step 404 may include generating a second color value vector associated with an exemplary second image. In an exemplary embodiment, an exemplary second color value vector may include a second array of a respective second set of three numerical color values of three respective color components of color of each respective chemical receptor of an exemplary array of chemical receptors 108 in an exemplary second image. In an exemplary embodiment, exemplary three-color components may include red, green, and blue. In an exemplary embodiment, generating an exemplary second color value vector may include extracting color values of blue, green, and red associated with each chemical receptor in an exemplary second image and forming an exemplary second array of exemplary respective second sets of extracted three color values respective to an exemplary array of chemical receptors 108. In an exemplary embodiment, extracting color values of blue, green, and red may be done using an image processing technique utilizing one or more processors. In an exemplary embodiment, an exemplary second color value vector may be formed utilizing one or more processors. In an exemplary embodiment, an exemplary second color may be formed using a relation defined by Equation 2 as follows:

V₂=[R₁₂G₁₂B₁₂ . . . Rᵢ₂Gᵢ₂Bᵢ₂ . . . Rₙ₂Gₙ₂Bₙ₂], Equation 2

Where, Rᵢ₂ is red component value of color of i^{th} chemical receptor of an exemplary array of chemical receptors 108 in an exemplary second image, Gᵢ₂ is green component value of color of i^{th} chemical receptor of an exemplary array of chemical receptors 108 in an exemplary second image, Bᵢ₂ is blue component value of color of i^{th} chemical receptor of an exemplary array of chemical receptors 108 in an exemplary second image, and n is total number of chemical receptors of an exemplary array of chemical receptors 108.

In further detail with respect to step 406, step 406 may include generating a difference color value vector by subtracting each color value of an exemplary first color value vector from a respective color value of an exemplary second color value vector. In an exemplary embodiment, an exemplary difference color value vector may be calculated using Equation 3 as follows:

ΔV=V₂−V₁=[ΔR₁ΔG₁ΔB₁ . . . ΔRᵢΔGᵢΔBᵢ . . . ΔRₙΔGₙΔBₙ], Equation 3

Where, ΔRᵢ, ΔGᵢ, and ΔBᵢ is defined by respective Equations 4 to 6 as follows:

ΔRᵢ=Rᵢ₂−Rᵢ₁, Equation 4

ΔGᵢ=Gᵢ₂−Gᵢ₁, Equation 5

ΔBᵢ=Bᵢ₂−Bᵢ₁, Equation 6

In further detail with respect to step 408, step 408 may include detecting COVID-19 infection status of an exemplary person based on an exemplary difference color value vector. In further detail with respect to step 408, step 408 may include detecting COVID-19 infection status of an exemplary person based on at least one of value level of one or more elements of an exemplary difference color value vector, a magnitude of an exemplary difference color value vector, and combinations thereof. In an exemplary embodiment, detecting COVID-19 infection status of an exemplary person based on an exemplary difference color value vector may include comparing each element of the difference color value vector with a respective element of three reference difference color value vectors and detecting the person is one of a COVID-19 infected patient, a healthy individual, or a cured individual after a COVID-19 infection. In an exemplary embodiment, the three reference difference color value vectors may include a first mean difference color value vector of a first plurality of difference color value vectors generated for an exemplary first respective set of images associated with a respective plurality of COVID-19 infected patients, a second mean difference color value vector of a second plurality of difference color value vectors generated for an exemplary second respective set of images associated with a respective plurality of healthy individuals, and a third mean difference color value vector of a third plurality of difference color value vectors generated for an exemplary third set of images associated with a respective plurality of cured individuals after a COVID-19 infection. In an exemplary embodiment, detecting an exemplary person is one of a COVID-19 infected patient, a healthy individual, or a cured individual after a COVID-19 infection may include detecting an exemplary person is infected by COVID-19 virus if a difference percentage between a value level of each element of the difference color value vector and a value level of the respective element of the first reference difference color value vector is less than 5%. In an exemplary embodiment, detecting an exemplary person is one of a COVID-19 infected patient, a healthy individual, or a cured individual after a COVID-19 infection may include detecting an exemplary person is healthy if a difference percentage between a value level of each element of the difference color value vector and a value level of the respective element of the second reference difference color value vector is less than 5%. In an exemplary embodiment, detecting an exemplary person is one of a COVID-19 infected patient, a healthy individual, or a cured individual after a COVID-19 infection may include detecting an exemplary person is cured after a COVID-19 infection if a difference percentage between a value level of each element of the difference color value vector and a value level of the respective element of the third reference difference color value vector is less than 5%. As used herein, a “difference percentage” between two values may include a relative difference of a first value of the two values to a second value of the two values.

In an exemplary embodiment, exemplary method 300 may further include generating the three reference difference color value vectors. In an exemplary embodiment, generating the three reference difference color value vectors may include generating three pluralities of difference color value vectors associated with three respective pluralities of COVID-19 infected patients, healthy individuals, and cured individuals after a COVID-19 infection and forming a respective reference difference color value vector for each respective plurality of difference color value vectors of three pluralities of difference color value vectors by calculating an average of respective elements of each plurality of difference color value vectors.

In an exemplary embodiment, generating each plurality of difference color value vectors of three pluralities of difference color value vectors may include preparing an exemplary first image before exposing an exemplary colorimetric sensor 100 to exhaled breath similar to steps 302 exemplary method 300, preparing an exemplary second image for each person of each exemplary plurality of COVID-19 infected patients, healthy individuals, and cured individuals by applying steps 303 to 306 of exemplary method 300, and generating a difference color value vector for each person using a process including steps 402 to 406 of exemplary method 400. In an exemplary embodiment, an exemplary reference difference color value vector (ΔV_{R}) may be generated using a relation defined by Equation 7 as follows:

$$Δ {V}_{R} = [ \frac{{\sum }_{1}^{P} Δ {R}_{1}}{P} \frac{{\sum }_{1}^{P} Δ {G}_{1}}{P} \frac{{\sum }_{1}^{P} Δ {B}_{1}}{P} … \frac{{\sum }_{1}^{P} Δ {R}_{i}}{P} \frac{{\sum }_{1}^{P} Δ {G}_{i}}{P} \frac{{\sum }_{1}^{P} Δ {B}_{i}}{P} … \frac{{\sum }_{1}^{P} Δ {R}_{n}}{P} \frac{{\sum }_{1}^{P} Δ {G}_{n}}{P} \frac{{\sum }_{1}^{P} Δ {B}_{n}}{P} ] , \qquad Equation 7$$

Where, P is total number of people of an exemplary plurality of people in a respective group of three groups of COVID-19 infected patients, healthy individuals, and cured individuals.

In an exemplary embodiment, detecting COVID-19 infection status of an exemplary person based on an exemplary difference color value vector may include calculating a magnitude of an exemplary difference color value vector and detecting COVID-19 infection status of an exemplary person by comparing the magnitude of an exemplary difference color value vector with a threshold value. In an exemplary embodiment, calculating the magnitude of an exemplary difference color value vector may include calculating Euclidean norm of an exemplary difference color value vector. In an exemplary embodiment, calculating the magnitude of an exemplary difference color value vector may be done using a relation defined by Equation 8 as follows:

|ΔV|={square root over (Σᵢ₌₁ⁿ((ΔRᵢ)²+(ΔGᵢ)²+(ΔBᵢ)²))}, Equation 8

Where, |ΔV| is magnitude of an exemplary difference color value vector.

In an exemplary embodiment, the threshold value may include a borderline value between a first range of magnitudes of an exemplary plurality of difference color value vectors associated with an exemplary respective plurality of COVID-19 patients and a second range of magnitudes of a plurality of difference color value vectors associated with a respective plurality of healthy individuals. In an exemplary embodiment, the threshold value may include an average of magnitudes of an exemplary plurality of difference color value vectors associated with an exemplary respective plurality of COVID-19 patients. In an exemplary embodiment, detecting the COVID-19 infection status of an exemplary person may include detecting an exemplary person is healthy if the magnitude of an exemplary difference color value vector is less than an exemplary threshold value or detecting an exemplary person is COVID-19 infected if the magnitude of an exemplary difference color value vector is more than an exemplary threshold value. In an exemplary embodiment, an exemplary threshold value may include a value of about 375.17. In an exemplary embodiment, an exemplary person is healthy if the magnitude of an exemplary difference color value vector is less than about 375.17. In an exemplary embodiment, an exemplary person is a COVID-19 infected patient if the magnitude of an exemplary difference color value vector is more than about 375.17.

Referring again to FIG. 3, in further detail with respect to step 308, detecting COVID-19 infection status of an exemplary person by analyzing color changes of an exemplary array of chemical receptors in an exemplary second image respective to an exemplary first image may include detecting an exemplary person is infected by COVID-19 virus if a change in color of at least one chemical receptor of a set of COVID-19 indicative chemical receptors is detected in the second image respective to the first image. In an exemplary embodiment, the set of COVID-19 indicative chemical receptors may include an aqueous solution of [Co(3,4-tmtppa)]⁴⁺ with a concentration of 1.5 mg·mL⁻¹, a solution of a mixture of indigo carmine and DWES in water with a volume ratio of indigo carmine:DWES equal to 4:1, a solution of a mixture of phenol red and DWES in ethanol with a volume ratio of phenol red:DWES equal to 4:1, a solution of a mixture of methyl red and TBAOH in ethanol with a volume ratio of methyl red:TBAOH equal to 4:1, a solution of Fe(III)TPPCl with a concentration of 6 mg mL⁻¹ in ethanol, and a solution of a mixture of bromocresol purple and PBA in ethanol with a volume ratio of bromocresol purple:PBA equal to 4:1.

In an exemplary embodiment, detecting COVID-19 infection status of an exemplary person by analyzing color changes of an exemplary array of chemical receptors in an exemplary second image respective to an exemplary first image may include detecting an exemplary person is healthy and not infected by COVID-19 virus if a change in color of at least one chemical receptor of a set of non-COVID-19 infection indicative chemical receptors is detected in the second image respective to the first image. In an exemplary embodiment, the set of non-COVID-19 infection indicative chemical receptors may include an aqueous solution of [Cu(2,3-tmtppa)]⁴⁺ with a concentration of 1.5 mg·mL⁻¹, a solution of a mixture of toluidine blue and DWES in water with a volume ratio of toluidine blue:DWES equal to 4:1, and a solution of a mixture of pararosaniline and TsOH in ethanol with a volume ratio of pararosanilin:TsOH equal to 4:1.

In an exemplary embodiment, detecting COVID-19 infection status of an exemplary person (step 308) may further include detecting a severity grade of COVID-19 infection of an exemplary person. In an exemplary embodiment, detecting a severity grade of COVID-19 infection of an exemplary person may be carried out by analyzing color changes of at least one severity indicator of a set of COVID-19 severity indicators of the array of chemical receptors in the second image respective to the first image. In an exemplary embodiment, the set of COVID-19 severity indicators may include the solution of the mixture of methyl red and TBAOH in ethanol with the volume ratio of methyl red:TBAOH equal to 4:1 and the solution of the mixture of bromocresol purple and PBA in ethanol with the volume ratio of bromocresol purple:PBA equal to 4:1.

In an exemplary embodiment, detecting a severity grade of COVID-19 infection of an exemplary person may include extracting two sets of three numerical color values of the at least one severity indicator in the first image and the second image, generating a difference color value vector associated with the at least one severity indicator by subtracting the two sets of three numerical color values from each other, calculating a magnitude of discoloration of the at least one severity indicator in the second image respective to the first image, and detecting severity grade of COVID-19 infection of an exemplary person based on the calculated magnitude of discoloration of the at least one severity indicator in the second image respective to the first image. In an exemplary embodiment, the three numerical color values may include respective values of three-color components of color of the at least one severity indicator. In an exemplary embodiment, the three-color components may include red, green, and blue.

In an exemplary embodiment, calculating the magnitude of discoloration of the at least one severity indicator in the second image respective to the first image may include calculating magnitude of the difference color value vector associated with the at least one severity indicator. In an exemplary embodiment, the magnitude of the difference color value vector associated with the at least one severity indicator may be calculated using a relation defined by Equation 9 as follows:

|ΔV|=\sqrt{(ΔR)²+(ΔG)²+(ΔB)²}, Equation 9

Where, |ΔV| is the magnitude of discoloration of the at least one severity indicator, ΔR is a difference between respective red color values in the first image and the second image, ΔG is a difference between respective green color values in the first image and the second image, and ΔB is a difference between respective blue color values in the first image and the second image.

In an exemplary embodiment, detecting the severity grade of COVID-19 infection of an exemplary person based on the calculated magnitude of discoloration of the at least one severity indicator in the second image respective to the first image may include one of detecting an exemplary person is mildly infected by COVID-19 virus, detecting an exemplary person is moderately infected by COVID-19 virus, detecting an exemplary person is severely infected by COVID-19 virus, and detecting an exemplary person is highly-severe infected by COVID-19 virus. In an exemplary embodiment, detecting the severity grade of COVID-19 infection of an exemplary person may include detecting the person is mildly infected by COVID-19 virus if the magnitude of discoloration of the at least one severity indicator is in a range of 0 to 91. In an exemplary embodiment, a mildly infection by COVID-19 virus may include a cycle threshold (CT) number in a range of 27 to 30. In an exemplary embodiment, an exemplary CT number may include a CT number for N gene obtained in a polymerase-chain-reaction (PCR) test applied to an exemplary person. In an exemplary embodiment, detecting the severity grade of COVID-19 infection of an exemplary person may include detecting the person is moderately infected by COVID-19 virus if the magnitude of discoloration of the at least one severity indicator is in a range of 92 to 121. In an exemplary embodiment, a moderately infection by COVID-19 virus may include a CT number in a range of 24 to 26. In an exemplary embodiment, detecting the severity grade of COVID-19 infection of an exemplary person may include detecting the person is severely infected by COVID-19 virus if the magnitude of discoloration of the at least one severity indicator is in a range of 122 to 145. In an exemplary embodiment, a severely infection by COVID-19 virus may include a CT number in a range of 19 to 23. In an exemplary embodiment, detecting the severity grade of COVID-19 infection of an exemplary person may include detecting the person is highly-severe infected by COVID-19 virus if the magnitude of discoloration of the at least one severity indicator is in a range of 145 to 176 or more. In an exemplary embodiment, a highly-severe infection by COVID-19 virus may include a CT number in a range of 15 to 18.

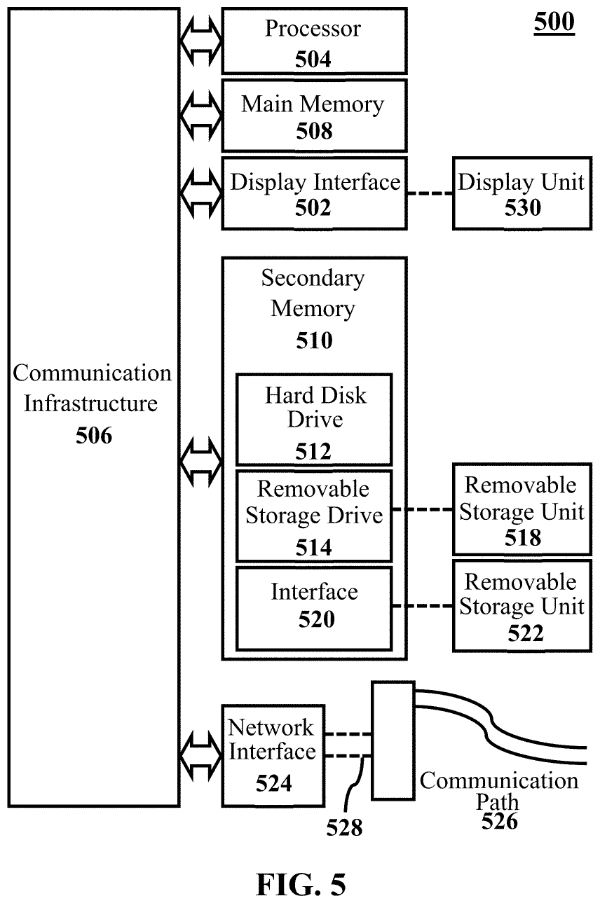

In an exemplary embodiment, one or more steps of methods 300 and 400 may be performed by processing unit 202. FIG. 5 shows an example computer system 500 in which an embodiment of the present disclosure, or portions thereof, may be implemented as computer-readable code, consistent with one or more exemplary embodiments of the present disclosure. For example, computer system 500 may include an example of processing unit 202 illustrated in FIG. 2, one or more steps of exemplary methods 300 and 400 presented in FIGS. 3 and 4 may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIG. 2.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores”.

An embodiment of the present disclosure is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative embodiments, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in exemplary methods 300 and 400 illustrated by FIGS. 3 and 4, discussed above. Accordingly, such computer programs represent controllers of computer system 500. Where an exemplary embodiment of methods 300 and 400 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Fabrication of a Colorimetric Sensor

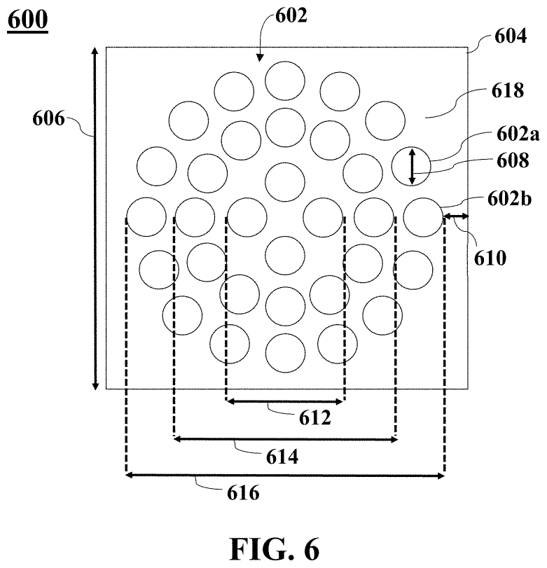

In this example, a colorimetric sensor similar to colorimetric sensors 100 and 206, described herein above, was fabricated. To design the colorimetric sensor, a pattern of 32 circular individual areas similar to array of individual areas 106 was drawn using a processing unit similar to processing unit 202. FIG. 6 shows an exemplary pattern 600 of an array of individual areas 602 of the colorimetric sensor, consistent with one or more exemplary embodiments of the present disclosure. Designed pattern 600 was printed on a square piece of filter paper 604 using a printer device. Square piece of filter paper 604 has a side dimension 606 of 2.5 cm. Pattern 600 includes 32 circular individual areas 602 where each circular individual area 602a has a diameter 608 of 0.2 cm. 32 circular individual areas 602 may be arranged in three circles with diameters 612, 614, and 616 respectively being equal to 0.9 cm, 1.54, and 2.25 cm. A distance 610 from an outer individual area 602b to side of square piece of filter paper 604 is about 0.125 cm. Region 618 including spaces of square piece of filter paper 604 among and around 32 circular individual areas 602 was coated with a hydrophobic printing ink after printing pattern 600 on square piece of filter paper 604, whereas 32 circular individual areas 602 remained bare and uncoated. After printing, square piece of filter paper 604 with pattern 600 printed thereon was heated in an oven at 200° C. for 45 min; thereby, resulting in blocking pores of region 618 with hydrophobic printing ink so that region 618 became a hydrophobic barrier among and around 32 circular individual areas 602 preventing spillover of a liquid from a first circular individual area of 32 circular individual areas 602 to a second circular individual area of 32 circular individual areas 602. Each circular individual area 602a was filled and coated with a relevant chemical receptor. About 0.2 μL of a solution containing each chemical receptor was dropped and deposited on each respective circular individual area 602a using a micropipette. Five categories of sensing elements including metalloporphyrazines, metalloporphyrins, organic dyes mixed with some additives, metal ion complexes, and functionalized gold nanoparticles (AuNPs) were used as chemical receptors. An array of 32 chemical receptors were coated respectively on 32 circular individual areas 602. The array of 32 chemical receptors included [Co(3,4-tmtppa)]⁴⁺, [Zn(3,4-tmtppa)]⁴⁺, [Cu(2,3-tmtppa)]⁴⁺, Fe(2,3-tmppa), bromocresol red+DWES, bromocresol purple+DWES, acridine orange+DWES, indigo carmine+DWES, toluidine blue+DWES, malachite green+DWES, phenol red+DWES, pararosaniline+DWES, pararosaniline+TsOH, thymol blue+TBAOH, methyl red+TBAOH, bromophenol blue+TBAOH, Mn(III)T(4-Cl)PP(OAc), Fe(III)TPPCl, Mn(III)T(4-OH)PP(OAc), Cu(II)TPP, Sn(II)TPP, V(IV)-Py, and Fe(III)-Py, Fe(II)-Py, Cu(II)-Py, Ni(II)-Py, bromocresol purple+PBA, bromopyrogallol red+PBA, methyl blue+PBA, GA-AuNPs, BSA-AuNPs, and PVP-AuNPs. For preparing the array of 32 chemical receptors, water soluble porphyrazines and porphyrines, mixtures of organic dyes with additives, metal ion complexes, and functionalized AuNPs were synthesized/prepared and dropped on respective circular individual areas. Porphyrazines, metal salts and dyes, including acridine orange, indigo carmine, and pyrocatechol violet were dissolved in deionized water. Ethanol was used to prepare solutions of porphyrins and color materials including bromophenol red, toluidine blue, malachite green, phenol red, pararosaniline hydrochloride, thymol blue, methyl red, bromophenol blue, bromopyrogallol red, methyl blue, and bromocresol purple. Then, a prepared mixture was filtered and an obtained solution was used for chemical receptors including DWES. DWES solution was obtained by adding 2.0 mL of H₂SO₄ to a mixture containing 0.4 g of 2,4-dinitrophenylhydrazine, 10.0 mL EtOH and 3.0 mL deionized water and stirring the resulting solution for 10 min. After filtration, an obtained pure yellow solution was collected. Organic dye solutions were individually mixed with additives of DWES, TsOH (2.0 M), TBAOH (concentrated), and PBA (2.0 M) based on an optimized protocol described herein below. Metal ion complexes were prepared by mixing 100.0 μL borate buffer (0.1 M and pH=9.0), 50 μL pyrocatechol violet (Py), and 50 μL of an aqueous solution of one of V (IV), Fe (III), Fe (II), Cu (II), Ni (II) ions. Concentrations of Py and metal ion solutions were equal which was adjusted at an optimized value as described herein below. Three pluralities of AuNPs functionalized with GA, BSA and PVP were synthesized and re-dispersed in deionized water for use as sensing elements in the colorimetric sensor. By using a laboratory freeze-dryer, the prepared NPs were dried.

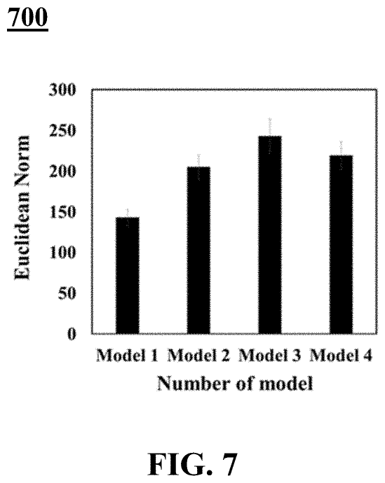

Furthermore, an optimization process was done to achieve optimum amount/concentration of each chemical receptor and optimum time period of interaction between exhaled breath and the array of chemical receptors; thereby, leading to the highest accuracy classification of the fabricated colorimetric sensor to discriminate patients from healthy individuals. In this regard, four test models similar to exemplary method 300 were designed and performed by varying amounts/ratios of chemical receptors. Amounts of chemical receptors used in each model are summarized in Table 1 while Model 1 and Model 4 contains the lowest and the highest amounts of chemical receptors, respectively. For each model, a magnitude of an exemplary difference color value vector (|ΔV|) of an exemplary array of chemical receptors was calculated as a response of the colorimetric sensor. FIG. 7 shows a chart 700 of response of the colorimetric sensor using four models described in Table 1 for optimizing concentration of each exemplary chemical receptor, consistent with one or more exemplary embodiments of the present disclosure. As may be seen in FIG. 7, the strongest interaction between exhaled breath metabolites and the colorimetric sensor components, together with the best sensor response is achieved by using Model 3 for preparing the chemical receptors solutions. Lower values of chemical receptors are not sufficient to complete reaction, and higher values prevent monitoring of changes arising from interactions.

TABLE 1
Model tests for optimization of concentration of chemical receptors

| Number of trial | Information |
|---|---|
| Model 1 | Porphyrazines (0.5 mg · mL⁻¹), Organic dyes* (1.0 mg · mL⁻¹), Porphyrins |
| | (2.0 mg · mL⁻¹), Metal complexes** (0.001M), NPs (1.0 mg · mL⁻¹) |
| Model 2 | Porphyrazines (1.0 mg · mL⁻¹), Organic dyes* (2.0 mg · mL⁻¹), Porphyrins |
| | (4.0 mg · mL⁻¹), Metal complexes** (0.005M), NPs (2.0 mg · mL⁻¹) |
| Model 3 | Porphyrazines (1.5 mg · mL⁻¹), Organic dyes* (3.0 mg · mL⁻¹), Porphyrins |
| | (6.0 mg · mL⁻¹), Metal complexes** (0.01M), NPs (3.0 mg · mL⁻¹) |
| Model 4 | Porphyrazines (2.0 mg · mL⁻¹), Organic dyes* (4.0 mg · mL⁻¹), Porphyrins |
| | (8.0 mg · mL⁻¹), Metal complexes** (0.05M), NPs (4.0 mg · mL⁻¹) |

*Organic dyes were mixed with additives with ratio of 1:1 V/V
**Metal complexes were dissolved in borate buffer (0.1M)

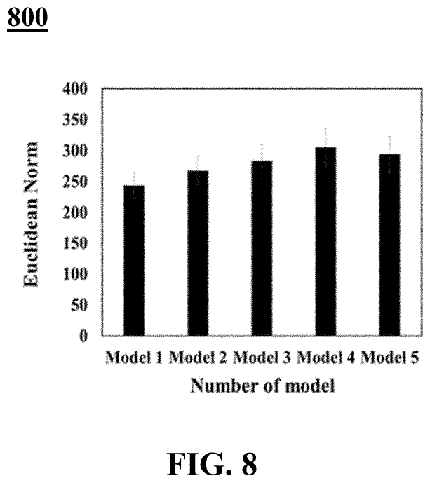

In a next step of optimization of chemical receptors amounts/concentrations, organic dyes were mixed with additives in different volume ratios of (1:1), (2:1), (3:1), (4:1), and (5:1), as summarized in Models 1-5 in Table 2. FIG. 8 shows a chart 800 of response of the colorimetric sensor using five models described in Table 2 for optimizing ratio of organic dyes to additives, consistent with one or more exemplary embodiments of the present disclosure. FIG. 8 shows that color changes of chemical receptors become more intense with increasing contribution of organic dye in a mixture of an organic dye with an additive. In this respect, the highest response is obtained using volume ratio of (4:1). However, it should be noted that the highest interaction does not occur at the lowest amount of additives because active sites of organic dyes are blocked using high amounts of these materials.

TABLE 2
Model tests for optimization of volume ratio of an organic dye to
an additive in a solution containing the organic dye and additive

| Number of trial | The volume ratio of (organic dye:additive*) |
|---|---|
| Model 1 | 1:1 |
| Model 2 | 2:1 |
| Model 3 | 3:1 |
| Model 4 | 4:1 |
| Model 5 | 5:1 |

*Additive is one of DWES, TsOH, TBAOH, and PBA

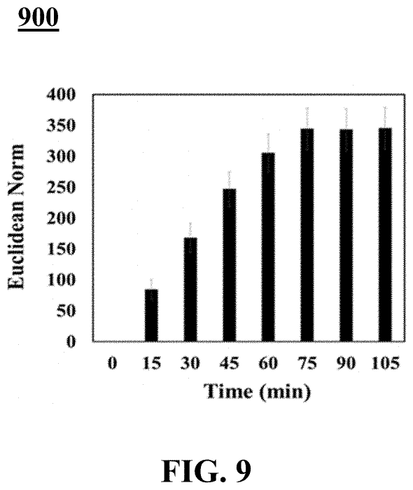

Moreover, an optimal time for incubation of chemical receptors with exhaled breath analytes was determined. Volatile metabolites must be given time to distribute throughout the colorimetric sensor surface, and interact with the array of chemical receptors. An analysis will be stopped once color changes of all chemical receptors are fixed, being indicative of completion of the reaction. FIG. 9 shows a chart 900 of response of the colorimetric sensor for different periods of time of incubation of exhaled breath and the array of chemical receptors, consistent with one or more exemplary embodiments of the present disclosure. FIG. 9 shows that about 75 min is required to carry out a complete interaction between the array of chemical receptors and exhaled breath.

### Example 2: COVID-19 Infection Diagnosis Using a Colorimetric Sensor Exposed to Exhaled Breath Metabolites

In this example, color changes of an exemplary array of chemical receptors of an exemplary colorimetric sensor similar to colorimetric sensor 100 (or 206) fabricated according to EXAMPLE 1 and optimal amounts obtained herein above was monitored and analyzed utilizing a method similar to exemplary method 300 to diagnose COVID-19 patients and differentiate them from healthy and cured individuals. 115 participants were men with an age range of 21-80 years were divided into three groups, including 60 COVID-19 patients, 55 healthy individuals, and 15 cured people after an infection with COVID-19 virus. Demographic data of the participants is shown in Table 3. The COVID-19 patients were examined by a pulmonologist and their disease was confirmed using chest imaging and real-time reverse-transcriptase polymerase-chain-reaction (rRT-PCR) results.

TABLE 3
Demographic data of the studied population

| Variable | Patient with Covid-19 | Healthy controls |
|---|---|---|
| Studied samples | 60 | 55 |
| Age | 52.15 ± 14.01 | 48.10 ± 11.15 |
| (Mean ± SD) | | |
| O₂ saturation*** | 91.29 (75-97) | 97.45 (96-99) |
| RT-PCR* | Positive (91.6%) | Negative (100%) |
| N gene*** | 24.30 (15-32) | |
| RdRp gene*** | 24.35 (15-34) | |
| Smoke | 5 | 15 |
| Comorbidities | | |
| Cardiovascular disease | 6 | 8 |
| Chronic kidney disease | 7 | 8 |
| Asthma | 1 | 1 |
| Diabetes | 11 | 9 |
| COPD** | 4 | 3 |
| Chronic liver disorder | 1 | 5 |
| Hypertension | 11 | 9 |
| Symptoms | | |
| Cough | 56 | |
| Dyspnea | 48 | |
| Headache | 53 | |
| Fever | 49 | |
| Myalgia | 43 | |
| Nausea and Diarrhea | 24 | |
| Anorexia | 38 | |
| Sneezing | 8 | |

*RT-PCR: Reverse transcription polymerase chain reaction
**COPD: Chronic obstructive pulmonary disease
***Data are represented as median and interquartile range.

The fabricated colorimetric sensor was pasted onto center of a flexible and mechanically stable plastic strip with size of 2.5 cm×5.5 cm and sides of the strip were covered with double-sided adhesive. A sterile package containing a colorimetric sensor pasted on the plastic strip, a sterile thin cloth mask, and a three-layer medical mask was given to each participant to put in front of his/her face as described herein above in connection with FIGS. 1C-1E. First, the thin cloth mask was placed on the face, the double-sided adhesive was then removed, and the colorimetric sensor was stuck to the thin cloth mask. Then, the colorimetric sensor was covered with the three-layer medical mask to prevent physical interference and environmental pollutions. The participant was asked to normally perform inhalation and exhalation cycles for a certain period of time (i.e., 75 minutes) without any external force. During this time, the participant could continue his daily activities (except sleeping and eating). Exhaled breath metabolites directly interacted with chemical receptors, resulting in discoloration of the chemical receptors. Color changes were observed by naked eye. Color change responses were recorded by a scanner, and recorded images from the array of chemical receptors were analyzed using a processing unit similar to processing unit 202. For each chemical receptor, outputs were three numerical values obtained from difference of mean values of R, G and B color elements. The difference value was calculated by subtracting RGB values of a first image recorded from the array of chemical receptors from RGB values of a second image recorded form array of chemical receptors reacted with exhaled breath. In total, a vector of 96 data points (32 chemical receptors×3 color elements) was obtained for each participant.

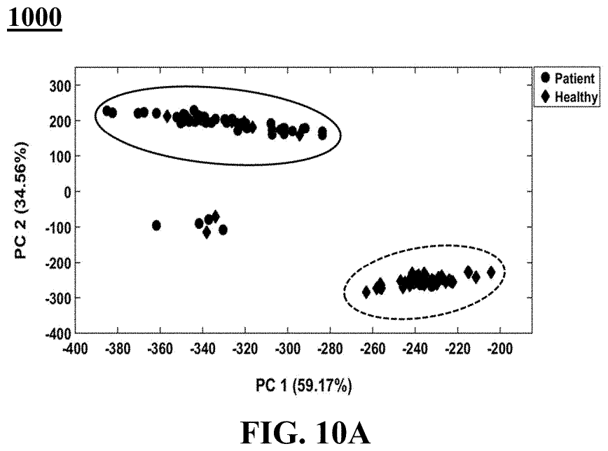

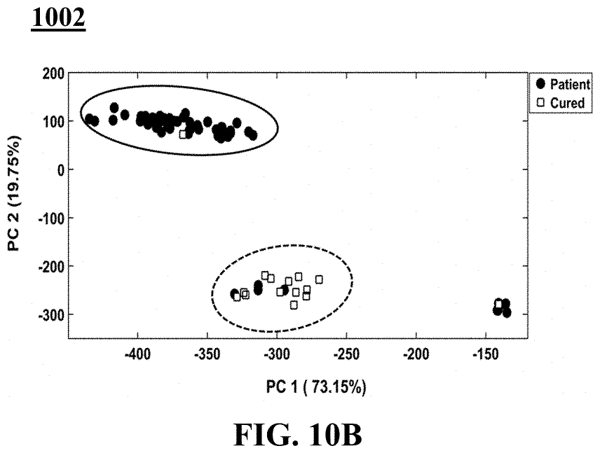

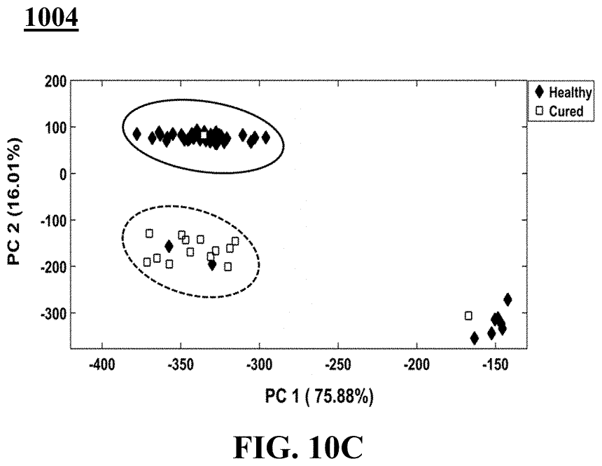

Evaluation of discrimination ability of exemplary colorimetric sensor was performed by principle component analysis-linear discriminate analysis (PCA-LDA). Image analysis data were aligned in three individual matrices and color difference data were collected in matrices with sizes of (115×96), (75×96), and (70×96) for patient-healthy, patient-cured, and healthy-cured classes, respectively. These data were inserted into PCA-LDA algorithm for cluster statistical analysis. FIGS. 10A-10C show respective charts 1000, 1002, and 1004 of score plots obtained by PCA-LDA analysis for classification of patients, healthy controls, and cured participants using an exemplary fabricated colorimetric sensor with optimized array of chemical receptors compositions after 75 min exposure of an exemplary array of chemical receptors to exhaled breath of participants, consistent with one or more exemplary embodiments of the present disclosure. FIG. 10A shows a chart 1000 of score plots for classification of patients and healthy controls, FIG. 10B shows a chart 1002 of score plots for classification of patients and cured individuals, and FIG. 10C shows a chart 1004 of score plots for classification of healthy controls and cured individuals.

The score plots depicted in FIGS. 10A-10C show that over about 92% of total data variances are distributed in space of two first principal components of PCA-LDA analysis. Shown diagrams indicate high accurate distinction between two classes of each matrix so that 47 patients, 46 healthy individuals, and 13 cured samples can be classified in their corresponding classes with sensitivities of 78.3%, 78.3%, and 86.3%, respectively. In detail, COVID-19 patients (60 samples) and healthy individuals (55 samples) were discriminated with 80.8% accuracy. Moreover, results showed that exhaled breath metabolites of cured and healthy participants is different from each other with accuracy of 84.3%. It should be noted that in some cases, improper transfer of chemical markers from mask cavities into sensor texture and low concentration of COVID-19 markers in exhaled breath profile due to mildness of disease in some patients may lead to adverse responses of exemplary colorimetric sensor.

Analysis of color changes of an exemplary array of chemical receptors after 75 minutes interaction with exhaled breath metabolites showed that while [Zn(3,4-tmtppa)]⁺⁴, Bromophenol red+DWES, and Cu(II)-Py respond to volatile metabolites of all tested samples, no color changes is observed for Fe(II) (2,3-tmppa), Acridine orange+DWES, Bromophenol blue+TBAOH, Cu (II) TPP, Sn (II) TPP, Fe (II)-Py, Ni (II)-Py, Bromopyrogallol red+PBA, Methyl blue+PBA, and all AuNPs. It was indicated that color of [Co (3,4-tmtppa)]⁺⁴ and [Cu (2,3-tmtppa)]⁺⁴ is changed in colorimetric profiles of patient and healthy samples, respectively. DWES-organic dyes, including bromocresol purple, indigo carmine, malachite green, and phenol red showed a high tendency towards volatile compounds in COVID-19 patient's exhaled breath. Markers such as toluidine blue and pararosaniline interact with metabolites of healthy individuals. Dyes combined with TsOH tend to interact with volatile markers in healthy individuals, whereas TBAOH-integrated indicators become discolored in the presence of COVID-19 infected breath samples. Among porphyrins, only Fe (III) TPPCl responds to COVID-19 patient metabolites. Both metalloporphyrins with central metal Mn and metal ion complexes of V (IV) and Fe(III) discolored in color profile of healthy individuals. Although Bromocresol purple+PBA is sensitive to chemical species of breath sample of COVID-19 patients, other PBA-organic dyes are not influenced by breath compositions of all three studied groups. Sensor response to volatile metabolites of cured samples was different from results obtained for healthy and infected volunteers. Chemical receptors including bromocresol purple+DWES, malachite green+DWES, pararosaniline+DWES, thymol blue+TBAOH, Mn(III)T(4-Cl)PP(OAc), Mn(III)T(4-OH)PP(OAc), V(IV)-Py, and Fe(III)-Py were discolored in color map of cured samples. Discoloration of some indicators of COVID-19 infection in color map of cured individuals may indicate that body's metabolic behavior is still affected by COVID-19 viral infection. Moreover, some dedicated sensing elements for healthy samples appear in color profile of cured participants, evidencing progress in treatment process as well as accession of normal situation.

Additionally, total response of exemplary colorimetric sensor was calculated for each tested individual by calculating Euclidean norm of a respective difference color value vector using Equation 8. Mean of responses for each of patient and healthy groups was equal to 387.88 (±33.11) and 361.29 (±22.34), respectively. For all patient and healthy individuals, total average was 375.17 (±31.33). Results confirmed that the mean value obtained by patients was higher than that by healthy ones and the difference of 26.59 between mean total responses of patients and healthy individuals was statistically significant (P-value<0.001). It may also be concluded that Euclidean norm of higher than total average value of 375.17 is an indicative of COVID-19 infection, whereas Euclidean norm for a healthy participant has a lower value than the total average.

### Example 3: Detecting Severity of COVID-19 Infection

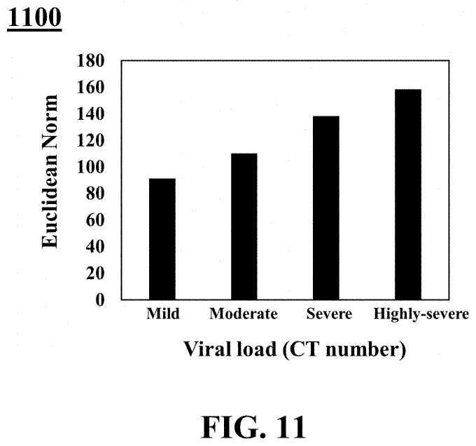

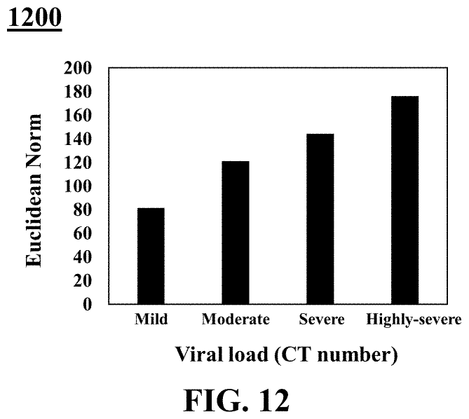

In this example, a colorimetric sensor similar to colorimetric sensors 100 and 206 and a system similar to exemplary system 200 was utilized through conducting a method similar to exemplary method 300 for detecting severity of COVID-19 infection based on a magnitude of an exemplary difference color value vector (or Euclidean norm of an exemplary difference color value vector) for each patient. COVID-19 patients tested in Example 2 hereinabove were divided into 5 groups with mild, moderate, severe, and highly-severe infection. Disease severity was determined by a pulmonologist using vital signs, symptoms, chest imaging, and rRT-PCR results. For severity detection, discoloration of an exemplary array of chemical receptors (or only one specific receptor) measured by Euclidean norm of an exemplary difference color value vector for one or more chemical receptor for each patient may be compared in accordance with five categorized severity levels. A relationship between response of two additionally chemical receptors methyl red+TBAOH and bromocresol purple+PBA and viral load value (extracted from rRT-PCR analysis based on number of N gene cycle threshold (CT)) was investigated. A viral load value (based on N gene cycle threshold (CT) value) obtained from a rRT-PCR analysis is a criterion for determining COVID-19 infection severity. Viral loads was classified to four numerical ranges of (28-32) for mild, (23-27) for moderate, (18-22) for severe, and (15-17) for highly-severe infection. FIG. 11 shows a chart 1100 of magnitude of an exemplary difference color value vector for chemical receptor methyl red combined with TBAOH (methyl red+TBAOH) versus severity levels of COVID-19 infection, consistent with one or more exemplary embodiments of the present disclosure. Additionally, FIG. 12 shows a chart 1200 of magnitude of an exemplary difference color value vector for chemical receptor bromocresol purple combined with PBA (bromocresol purple+PBA) versus severity levels of COVID-19 infection, consistent with one or more exemplary embodiments of the present disclosure. As is clear in FIGS. 11 and 12, response of chemical receptors methyl red+TBAOH and bromocresol purple+PBA shows an increasing trend with increasing viral load value. As depicted in Table 4, an accurate relationship between discoloration of methyl red+TBAOH and bromocresol purple+PBA, and COVID-19 infection severity is observed due to high Pearson coefficient (0.946) and low P (<0.001) values.

TABLE 4
Correlation of COVID-19 infection severity and viral load value with
response of special chemical receptors for the patient population

| | | Correlation results | |
|---|---|---|---|
| | | Pearson | |
| Variable | Chemical receptor | correlation | p-value |
| Viral load value | methyl red + TBAOH | 0.922 | <0.001 |
| | bromocresol purple + PBA | 0.931 | <0.001 |

### Example 4: Diagnosis of Comorbidities Alone with COVID-19 Infection

A person who is suspected to be infected with COVID-19 virus may be smoker or has other non-COVID diseases such as cardiovascular disease, chronic kidney disease, asthma, diabetes, chronic obstructive pulmonary disease, chronic liver disease, and hypertension. Metabolites of these diseases can also appear in breath profile. Volatile compounds such as acetonitrile and furan derivatives (for smokers), amines and ammonia (for people with chronic kidney disease), acetone, ethanol and methyl nitrate (for diabetics), and isoprene, 4,7-dimethyl-undecane, 2,6-dimethyl-heptane, acetaldehyde, 2-butyl octanol and methyl isobutyrate (for people with lung disorders) may be effective metabolites for differentiating between the relevant disease and normal cases. For participants of EXAMPLES 2 and 3 described herein above, corresponding comorbidities are listed in Table 3. In this example, potential of an exemplary colorimetric sensor, system and method for screening smokers and people with cardiovascular, chronic kidney, asthma, diabetes, chronic obstructive pulmonary, chronic liver and hypertension diseases was investigated utilizing exemplary colorimetric sensor, system, and method of EXAMPLES 2 and 3. Exemplary colorimetric sensor was exposed to exhaled breath of people with these diseases. Colorimetric response of exemplary colorimetric sensor exposed to exhaled breath of patients with COVID-19, being smokers or having kidney, diabetes, or lung disorders showed an interaction between exhaled breath of people with diabetes and an exemplary array of chemical receptors of the colorimetric sensor. Chemical receptor Bromopyrogallol red+PBA discolored specifically in an exemplary second image associated with diabetics. Kidney disease metabolites tend to interact with Bromophenol blue+TBAOH and BSA-AuNPs. Acridine orange+DWES responds to volatile compounds in smokers. Furthermore, color of Sn (II) TPP and Fe (II)-Py changes in color difference maps of participants with asthma and COPD. A diagnosis ability of the colorimetric sensor for detection of these diseases is determined to be about 85.0% for smokers, 86.6% for kidney disease, 75.0% for diabetes, and 75.0% for lung disorders. It was observed that metabolites of other diseases do not interact with an exemplary set of COVID-19 indicative chemical receptors that respond to COVID-19 related markers. Also, a specified chemical receptor discolors in the presence of metabolites of a particular disease. Thus, an exemplary colorimetric sensor may be utilized for a unique response for a certain disease.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments. This is for purposes of streamlining the disclosure, and is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

While various embodiments have been described, the description is intended to be exemplary, rather than limiting and it will be apparent to those of ordinary skill in the art that many more embodiments and embodiments are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

What is claimed is:

1- A system for detecting COVID-19 infection, comprising:
a colorimetric sensor, comprising:
an array of chemical receptors deposited on a respective array of individual areas of a hydrophilic paper substrate, the array of chemical receptors configured to be exposed to exhaled breath of a person suspected to be infected by COVID-19 virus, the array of chemical receptors comprising:
an array of metalloporphyrazines solutions;
an array of organic dyes solutions;
an array of metalloporphyrins solutions;
an array of metal ion complexes solutions; and
an array of functionalized gold nanoparticles (AuNPs) dispersions;
an image-capturing device, configured to capture an image from the array of chemical receptors; and
a processing unit electrically connected to the image-capturing device, the processing unit comprising:
a memory having processor-readable instructions stored therein; and
a processor configured to access the memory and execute the processor-readable instructions, which, when executed by the processor configures the processor to perform a method, the method comprising:
capturing, utilizing the image-capturing device, a first image from the array of chemical receptors before exposure to exhaled breath of the person;
capturing, utilizing the image-capturing device, a second image from the array of chemical receptors after at least 75 minutes of exposure to the exhaled breath of the person; and
detecting a COVID-19 infection status of the person by analyzing color changes of the array of chemical receptors in the second image respective to the first image, comprising:
generating a first color value vector associated with the first image, the first color value vector comprising a first array of a respective first set of three numerical color values of three respective color components of color of each respective chemical receptor of the array of chemical receptors in the first image, the three-color components comprising red, green, and blue;
generating a second color value vector associated with the second image, the second color value vector comprising a second array of a respective second set of three numerical color values respective to the three-color components of color of each respective chemical receptor of the array of chemical receptors in the second image;
generating a difference color value vector by subtracting each color value of the first color value vector from a respective color value of the second color value vector;
calculating a magnitude of the difference color value vector; and
detecting the COVID-19 infection status of the person, comprising:
detecting the person being healthy responsive to the magnitude of the difference color value vector being less than 375.17; or
detecting the person being COVID-19 infected responsive to the magnitude of the difference color value vector being more than 375.17.

2- A system for detecting COVID-19 infection, comprising:
a colorimetric sensor, comprising:
an array of chemical receptors deposited on a respective array of individual areas of a hydrophilic paper substrate, the array of chemical receptors configured to be exposed to exhaled breath of a person suspected to be infected by COVID-19 virus, the array of chemical receptors comprising:
an array of metalloporphyrazines solutions;
an array of organic dyes solutions;
an array of metalloporphyrins solutions;
an array of metal ion complexes solutions; and
an array of functionalized gold nanoparticles (AuNPs) dispersions;
an image-capturing device, configured to capture an image from the array of chemical receptors; and
a processing unit electrically connected to the image-capturing device, the processing unit comprising:
a memory having processor-readable instructions stored therein; and
a processor configured to access the memory and execute the processor-readable instructions, which, when executed by the processor configures the processor to perform a method, the method comprising:
capturing, utilizing the image-capturing device, a first image from the array of chemical receptors before exposure to exhaled breath;
capturing, utilizing the image-capturing device, a second image from the array of chemical receptors after at least 75 minutes of exposure to exhaled breath; and
detecting a COVID-19 infection status of the person by analyzing color changes of the array of chemical receptors in the second image respective to the first image, comprising:
generating a first color value vector associated with the first image, the first color value vector comprising a first array of a respective first set of three numerical color values of three respective color components of color of each respective chemical receptor of the array of chemical receptors in the first image, the three-color components comprising red, green, and blue;
generating the second color value vector associated with the second image, the second color value vector comprising a second array of a respective second set of three numerical color values respective to the three-color components of color of each respective chemical receptor of the array of chemical receptors in the second image;
generating the difference color value vector by subtracting each color value of the first color value vector from a respective color value of the second color value vector; and
detecting the COVID-19 infection status of the person based on the difference color value vector.

3- The system of claim 2, wherein each metalloporphyrazine solution of the array of metalloporphyrazines solutions comprises an aqueous solution of one of tetramethyl tetra-3,4-pyridinoporphyrazinato cobalt(II) ([Co(3,4-tmtppa)]⁴⁺), tetramethyl tetra-2,3-pyridinoporphyrazinato copper(II) ([Cu(2,3-tmtppa)]⁴⁺), tetramethyl tetra-3,4-pyridinoporphyrazinato zinc(II) ([Zn(3,4-tmtppa)]⁴⁺), and tetramethyl tetra-3,4-pyridinoporphyrazinato iron(II) (Fe(2,3-tmppa)) with a concentration of 1.5 mg mL⁻¹.

4- The system of claim 2, wherein each metalloporphyrin solution of the array of metalloporphyrins solutions comprises a solution of one of [meso-tetraphenylporphyrin] iron(III) chloride (Fe(III)TPPCl), meso-tetrakis(4-chlorophenyl) porphyrin-manganese(III) acetate (Mn(III)T(4-Cl)PP(OAC)), eso-tetrakis(4-hydroxyphenyl) porphyrin-manganese(III) acetate (Mn(III)T(4-OH)PP(OAC)), [meso-tetraphenylporphyrin]-copper(II) (Cu(II)TPP), and [meso-tetraphenylporphyrin]-tin(II) (Sn(II)TPP) with a concentration of 6 mg mL⁻¹ in ethanol.

5- The system of claim 2, wherein each organic dye solution of the array of organic dyes solutions comprises:
an organic dye comprising at least one of bromophenol red, bromocresol purple, acridine orange, indigo carmine, toluidine blue, malachite green, phenol red, pararosaniline, thymol blue, methyl red, bromophenol blue, bromopyrogallol red, methyl blue, and combinations thereof;
an additive comprising at least one of DWES, phenylboronic acid (PBA), p-toluenesulfonic acid monohydrate (TsOH), tetrabutylammonium hydroxide (TBAOH), and combinations thereof,
wherein DWES is a mixture of 2,4-dinitrophenylhydrazine, deionized water, Ethanol (EtOH), and H₂SO₄; and
a solvent comprising at least one of water, ethanol, and combinations thereof,
wherein a mixture of the organic dye and the additive with volume ratio of organic dye:additive equal to 4:1 is dissolved in the solvent.

6- The system of claim 2, wherein each functionalized AuNPs dispersion of the array of functionalized AuNPs dispersions comprises 3 mg mL⁻¹ of a plurality of AuNPs functionalized with at least one of bovine serum albumin (BSA), gallic acid (GA), polyvinyl pyrrolidone (PVP), and combinations thereof dispersed in deionized water.

7- The system of claim 2, wherein each metal ion complex solution of the array of metal ion complex solution comprises a solution of at least one metal ion complex with a concentration of 0.01 mol L⁻¹ in a buffer solution with pH value of 9.0.

8- The system of claim 7, wherein the at least one metal ion complex comprises a complex of pyrocatechol violet (Py) with at least one of V (IV) ions, Fe (III) ions, Fe (II) ions, Cu (II) ions, Ni (II) ions, and combinations thereof.

9- The system of claim 2, wherein each of the first color value vector and the second color value vector is defined by:
Vⱼ=[R₁ⱼG₁ⱼB₁ⱼ . . . RᵢⱼGᵢⱼBᵢⱼ . . . RₙⱼGₙⱼBₙⱼ],
wherein:
j is equal to 1 for the first image and 2 for the second image;
Rᵢⱼ comprises red component value of color of i^{th} chemical receptor of the array of chemical receptors in the j^{th} image;
Gᵢⱼ comprises green component value of color of i^{th} chemical receptor of the array of chemical receptors in the j^{th} image;
Bᵢⱼ comprises blue component value of color of i^{th} chemical receptor of the array of chemical receptors in the j^{th} image; and
n comprises total number of chemical receptors of the array of chemical receptors.

10- The system of claim 9, wherein the difference color value vector is defined by:
ΔV=[ΔR₁ΔG₁ΔB₁ . . . ΔRᵢΔGᵢΔBᵢ . . . ΔRₙΔGₙΔBₙ],
wherein:
ΔRᵢ is defined by a relation of ΔRᵢ=Rᵢ₂−Rᵢ₁;
ΔGᵢ is defined by a relation of ΔGᵢ=Gᵢ₂−Gᵢ₁; and
ΔBᵢ is defined by a relation of ΔBᵢ=Bᵢ₂−Bᵢ₁.

11- The system of claim 10, wherein detecting the COVID-19 infection status of the person based on the difference color value vector comprises:
calculating a magnitude of the difference color value vector using a relation defined by:

|ΔV|=\sqrt{Σᵢ₌₁ⁿ(ΔRᵢ)²+(ΔGᵢ)²+(ΔBᵢ)²},
wherein |ΔV| is the magnitude of the difference color value vector; and
detecting the COVID-19 infection status of the person, comprising:
detecting the person being healthy responsive to the magnitude of the difference color value vector being less than a threshold value; or
detecting the person being COVID-19 infected responsive to the magnitude of the difference color value vector being more than the threshold value.

12- The system of claim 11, wherein the threshold value comprises a value of 375.17.

13- The system of claim 10, wherein detecting the COVID-19 infection status of the person based on the difference color value vector comprises:
comparing each element of the difference color value vector with a respective element of three reference difference color value vectors, the three reference difference color value vectors comprising:
a first mean difference color value vector of a first plurality of difference color value vectors generated by exposing the colorimetric sensor to exhaled breath of a respective plurality of COVID-19 patients;
a second mean difference color value vector of a second plurality of difference color value vectors generated by exposing the colorimetric sensor to exhaled breath of a respective plurality of healthy individuals; and
a third mean difference color value vector of a third plurality of difference color value vectors generated by exposing the colorimetric sensor to exhaled breath of a respective plurality of cured individuals after a COVID-19 infection; and
detecting the person being one of a COVID-19 infected patient, a healthy individual, or a cured individual after a COVID-19 infection, comprising:
detecting the person being infected by COVID-19 virus responsive to a difference percentage between each element of the difference color value vector and the respective element of the first reference difference color value vector being less than 5%;
detecting the person being healthy responsive to a difference percentage between each element of the difference color value vector and the respective element of the second reference difference color value vector being less than 5%; or
detecting the person being cured after a COVID-19 infection responsive to a difference percentage between each element of the difference color value vector and the respective element of the third reference difference color value vector being less than 5%.

14- The system of claim 13, wherein the method further comprises generating the three reference difference color value vectors, comprising:
generating three pluralities of difference color value vectors by exposing the colorimetric sensor to exhaled breath of three respective groups, the three groups comprising:
a plurality of COVID-19 patients;
a plurality of healthy individuals; and
a plurality of cured individuals after a COVID-19 infection; and
forming a respective reference difference color value vector for each plurality of difference color value vectors of three pluralities of difference color value vectors by calculating an average of respective elements of each plurality of difference color value vectors.

15- A system for detecting COVID-19 infection, comprising:
a colorimetric sensor, comprising:
an array of chemical receptors deposited on a respective array of individual areas of a hydrophilic paper substrate, the array of chemical receptors configured to be exposed to exhaled breath of a person suspected to be infected by COVID-19 virus, the array of chemical receptors comprising:
an array of metalloporphyrazines solutions;
an array of organic dyes solutions;
an array of metalloporphyrins solutions;
an array of metal ion complexes solutions; and
an array of functionalized gold nanoparticles (AuNPs) dispersions;
an image-capturing device, configured to capture an image from the array of chemical receptors; and
a processing unit electrically connected to the image-capturing device, the processing unit comprising:
a memory having processor-readable instructions stored therein; and
a processor configured to access the memory and execute the processor-readable instructions, which, when executed by the processor configures the processor to perform a method, the method comprising:
capturing, utilizing the image-capturing device, a first image from the array of chemical receptors before exposure to exhaled breath;
capturing, utilizing the image-capturing device, a second image from the array of chemical receptors after at least 75 minutes of exposure to exhaled breath; and
detecting a COVID-19 infection status of the person by analyzing color changes of the array of chemical receptors in the second image respective to the first image.

16- The system of claim 15, wherein detecting the COVID-19 infection status of the person comprises detecting the person being infected by COVID-19 virus responsive to detecting a change in color of at least one chemical receptor of a set of COVID-19 indicative chemical receptors in the second image respective to the first image, the set of COVID-19 indicative chemical receptors comprising:
an aqueous solution of [Co(3,4-tmtppa)]⁴⁺ with a concentration of 1.5 mg·mL⁻¹;
a solution of a mixture of indigo carmine and DWES in water with a volume ratio of indigo carmine:DWES equal to 4:1;
a solution of a mixture of phenol red and DWES in ethanol with a volume ratio of phenol red:DWES equal to 4:1;
a solution of a mixture of methyl red and TBAOH in ethanol with a volume ratio of methyl red:TBAOH equal to 4:1;
a solution of Fe(III)TPPCl with a concentration of 6 mg mL⁻¹ in ethanol; and
a solution of a mixture of bromocresol purple and PBA in ethanol with a volume ratio of bromocresol purple:PBA equal to 4:1.

17- The system of claim 16, wherein detecting the COVID-19 infection status of the person further comprises detecting a severity grade of COVID-19 infection of the person by analyzing color changes of at least one severity indicator of a set of COVID-19 severity indicators of the array of chemical receptors in the second image respective to the first image, the set of COVID-19 severity indicators comprising:
the solution of the mixture of methyl red and TBAOH in ethanol with the volume ratio of methyl red:TBAOH equal to 4:1; and
the solution of the mixture of bromocresol purple and PBA in ethanol with the volume ratio of bromocresol purple:PBA equal to 4:1.

18- The system of claim 17, wherein detecting the severity grade of COVID-19 infection of the person comprises:
extracting two sets of three numerical color values of the at least one severity indicator in the first image and the second image, the three numerical color values comprising respective values of three-color components of color of the at least one severity indicator, the three-color components comprising red, green, and blue;
generating a difference color value vector associated with the at least one severity indicator by subtracting the two sets of three numerical color values from each other;
calculating a magnitude of discoloration of the at least one severity indicator in the second image respective to the first image by calculating magnitude of the difference color value vector associated with the at least one severity indicator defined by:

|ΔV|=\sqrt{(ΔR)²+(ΔG)²+(ΔB)²},
wherein:

|ΔV| is the magnitude of discoloration of the at least one severity indicator;
ΔR is a difference between red color values in the first image and the second image;
ΔG is a difference between green color values in the first image and the second image; and
ΔB is a difference between blue color values in the first image and the second image; and
detecting the severity grade of COVID-19 infection of the person, comprising:
detecting the person being mild infected by COVID-19 virus responsive to the magnitude of discoloration of the at least one severity indicator being in a range of 0 to 91, a mild infection by COVID-19 virus comprising a cycle threshold (CT) number for N gene obtained in a polymerase-chain-reaction (PCR) test applied to the person being in a range of 27 to 30;
detecting the person being moderately infected by COVID-19 virus responsive to the magnitude of discoloration of the at least one severity indicator being in a range of 92 to 121, a moderate infection by COVID-19 virus comprising the CT number being in a range of 24 to 26;
detecting the person being severely infected by COVID-19 virus responsive to the magnitude of discoloration of the at least one severity indicator being in a range of 122 to 145, a severe infection by COVID-19 virus comprising the CT number being in a range of 19 to 23; and
detecting the person being highly-severe infected by COVID-19 virus responsive to the magnitude of discoloration of the at least one severity indicator being in a range of 146 to 176, a highly-severe infection by COVID-19 virus comprising the CT number being in a range of 15 to 18.

19- The system of claim 15, wherein detecting the COVID-19 infection status of the person comprises detecting the person being healthy responsive to detecting a change in color of at least one chemical receptor of a set of non-COVID-19 infection indicative chemical receptors in the second image respective to the first image, the set of non-COVID-19 infection indicative chemical receptors comprising:
an aqueous solution of [Cu(2,3-tmtppa)]⁴⁺ with a concentration of 1.5 mg·mL⁻¹;
a solution of a mixture of toluidine blue and DWES in water with a volume ratio of toluidine blue:DWES equal to 4:1; and
a solution of a mixture of pararosaniline and TsOH in ethanol with a volume ratio of pararosaniline:TsOH equal to 4:1.
